(19) United States
(12) Patent Application Publication
(10) Pub. No.: US 2025/0041448 A1
(43) Pub. Date: Feb. 6, 2025

(54) Gene Therapy of Fibroblast Growth Factor 23 Related Hypophosphatemic Diseases

(71) Applicants: Genethon, Evry-Courcouronnes (FR); Institut National de la Sante et de la Recherche Medicale, Paris (FR); Universite d'Evry Val d'Essonne, Paris (FR)

(72) Inventors: Giuseppe RONZITTI, Fontainebleau (FR); Louisa JAUZE, Vitry sur Seine (FR); Séverine CHARLES, Evry-Courcouronnes (FR); Federico MINGOZZI, Philadelphia, PA (US)

(73) Assignees: Genethon, Evry-Courcouronnes (FR); Institut National de la Sante et de la Recherche Medicale, Paris (FR); Universite d'Evry Val d'Essonne, Evry (FR)

(21) Appl. No.: 17/603,355

(22) PCT Filed: Apr. 20, 2020
(86) PCT No.: PCT/EP2020/061015

§ 371 Date: Oct. 13, 2021

(30) Foreign Application Priority Data
Apr. 19, 2019 (EP) 19305518.3
Jun. 17, 2019 (EP) 19305768.4

(51) Int. Cl. A61K 48/00; A61K 38/18; A61P 7/00; C12N 15/86

(52) U.S. Cl. CPC A61K 48/0058; A61K 38/1825; A61P 7/00; C12N 15/86; C12N 2740/15043; C12N 2750/14143

(57) ABSTRACT

The invention relates to a nucleic acid construct for gene therapy of FGF-23 related hypophosphatemic diseases, in particular gene therapy directed to muscle, liver or hematopoietic tissue, more particularly liver tissue. The invention relates also to a vector comprising the nucleic acid construct, and their use for the treatment of FGF-23 related hypophosphatemic diseases, in particular XLH, by gene therapy.

### FIELD OF THE INVENTION

The invention is in the field of gene therapy of FGF-23 related hypophosphatemic diseases, in particular X-linked hypophosphatemia (XLH). The invention relates to a nucleic acid construct for gene therapy of FGF-23 related hypophosphatemic diseases, in particular gene therapy directed to muscle, liver or hematopoietic tissue, more particularly liver tissue. The invention also relates to a vector comprising the nucleic acid construct, and their use for the treatment of FGF-23 related hypophosphatemic diseases, in particular XLH, by gene therapy.

### BACKGROUND OF THE INVENTION

Fibroblast growth factor 23 (FGF-23 or FGF23) is a phosphaturic hormone produced by bone, which works by binding to Klotho-FGF receptor complexes. Excessive activity of FGF23 results in hypophosphatemic diseases, including various genetic diseases such as X-linked hypophosphatemia (XLH) and Autosomal dominant or recessive hypophosphatemic rickets (ADHR, ADHR1, ADHR2), and acquired diseases such as Tumor-induced osteomalacia (TIO), and chronic kidney disease-mineral and bone disorder (CKD-MBD) (reviewed in Seiji Fukumoto, Calcif. Tissue Int., 2016, 98, 334-340).

X-linked hypophosphatemia (XLH, OMIM #307800) clinical manifestation ranges from isolated hypophosphatemia to severe lower-extremity bowing. The disease appears in the first two years of life with lower-extremity bowing. In adults, enthesopathy (calcification of the tendons) associated with joint pain, spontaneous dental abscesses and sensorineural hearing loss have been reported. XLH is due to mutations in the phosphate-regulating neutral endopeptidase (PHEX) gene that induce an increase in the FGF23 circulating levels. The increased FGF23 function leads to a downregulation of the sodium-phosphate co-transporter in kidney. The co-transporter located in the renal proximal tubule mediates the re-adsorption of phosphate from urines. Its downregulation results in poor re-adsorption of phosphate and lower phosphate levels in blood. Additionally, the increase in FGF23 is associated with impaired 1,25(OH)2 vitamin D synthesis and increased degradation. Decreased blood phosphate levels and low vitamin D levels lead to defective bone mineralization and fractures.

Classic XLH treatment consists of oral phosphate and high-dose calcitriol, the active form of vitamin D. In fact, the response to i.v. phosphate therapy is sometimes unpredictable and complications include “overshoot” hyperphosphatemia, hypocalcemia and metastatic calcification and parenteral regimens are not practical for chronic disorders. However, oral therapy requires high doses, which frequently leads to diarrhea or gastric irritation and replacement therapy alone is never adequate when there is a significant renal phosphate wasting. Therefore, novel strategies for the treatment of FGF23-related hypophosphatemia are needed.

An animal model of the disease exists that derives from a natural deletion of the PHEX gene, the HypDuk model. This model recapitulates most of the disease manifestations, with lower phosphate levels in blood and impaired bone growth. Different therapeutic strategies have been tested in HypDuk mice. One approach uses anti-FGF23 neutralizing antibodies. Recently, a monoclonal antibody anti-FGF23 has been approved for the treatment of the pediatric form of XLH (Crysvita®, Ultragenyx). Another strategy consists in the use of a truncated form of the human FGF23 able to bind to the FGF23 receptor without inducing the intracellular cascade of activation that results from a functional interaction between the receptor and FGF23. This truncated FGF23 can be used as a competitor to reduce the increased FGF23 function observed in XLH (Goetz R. et al., PNAS, 2009, 107, 407-412).

No gene therapy for FGF-23 related hypophosphatemic diseases such as XLH has been reported so far. Therefore, there is a need for a gene therapy approach for the treatment of FGF-23 related hypophosphatemic diseases, in particular XLH.

### SUMMARY OF THE INVENTION

The inventors have engineered a nucleic acid construct and derived AAV vector for gene therapy of FGF-23 related hypophosphatemic diseases. After a single injection of this AAV vector in HypDuk mice, a normalized gain of weight, body size, tail length and circulating phosphate was observed in treated animals. The correction of the disease at biochemical, macroscopic and functional levels observed after a single injection of this AAV vector demonstrates the enhanced potency of a gene therapy approach based on this nucleic acid construct and derived vector, in particular AAV vector, for the treatment of FGF-23 related hypophosphatemic diseases.

Therefore, the invention relates to a nucleic acid construct for gene therapy of FGF-23 related hypophosphatemic diseases, which codes for a FGF23 fusion protein comprising:
(a) a signal peptide,
(b) a FGF23 C-terminal peptide which binds the FGFR/klotho complex,
(c) a cleavable linker, and
(d) a protein stabilizing moiety,
   wherein the signal peptide is at the N-terminus of the fusion protein and the FGF23 C-terminal peptide and protein stabilizing moiety are separated by the cleavable linker.

In some embodiments, the FGF23 C-terminal peptide comprises a sequence from any one of positions 175 to 189 to any one of positions 203 to 251 of SEQ ID NO: 1 or a sequence having at least 70%, 75%, 80%, 85%, 90%, 91%, 92%, 93%, 94%, 95%, 96%, 97%, 98%, or 99% identity with said sequence.

In some embodiments, the FGF23 C-terminal peptide comprises the RXXR motif in positions 176 to 179 of SEQ ID NO: 1.

In some preferred embodiments, the FGF23 C-terminal peptide comprises the sequence SEQ ID NO: 2 or a sequence having at least 70%, 75%, 80%, 85%, 90%, 91%, 92%, 93%, 94%, 95%, 96%, 97%, 98%, or 99% identity with said sequence.

In some embodiments, the signal peptide comprises a sequence selected from the group consisting of SEQ ID NO: 3 to 8; preferably SEQ ID NO: 7.

In some embodiments, the protein stabilizing moiety is human serum albumin, preferably comprising the sequence SEQ ID NO: 9.

In some embodiments, the cleavable linker comprises the sequence SEQ ID NO: 10.

In some preferred embodiments, the nucleic acid construct codes for a FGF23 protein comprising the sequence SEQ ID NO: 12 or 52 or a sequence having at least 85%, 90%, 91%, 92%, 93%, 94%, 95%, 96%, 97%, 98%, or 99% identity with any one of said sequences.

In some embodiments, the nucleic acid construct is codon optimized for expression in human.

In some preferred embodiments, the nucleic acid construct comprises the sequence SEQ ID NO: 13, 51 or 57 or a sequence having at least 80%, 85%, 90%, 91%, 92%, 93%, 94%, 95%, 96%, 97%, 98%, or 99% identity with any one of said sequences.

In some embodiments, the nucleic acid construct comprises an expression cassette wherein the coding sequence is operably linked to at least a promoter that is functional in the individual's target cells or tissue, in particular muscle, liver or hematopoietic cells or tissue. In some particular embodiments, the promoter is a liver-specific promoter, preferably human alpha-1 antitrypsin promoter.

In some embodiments, the nucleic acid construct further comprises one or more control elements selected from the group consisting of: an enhancer associated to the promoter, preferably human ApoE control region; an intron placed between the promoter and the coding sequence, preferably a modified HBB2 intron of SEQ ID NO: 17 or a modified FIX intron of SEQ ID NO: 19; and a transcription termination signal, preferably bovine growth hormone polyadenylation signal.

In some embodiments, the nucleic acid construct comprises or consists of DNA.

In other embodiments, the nucleic acid construct comprises or consists of RNA.

The invention also relates to a vector for gene therapy comprising the nucleic acid construct according to the invention.

In some embodiments, the vector is a viral vector, in particular an AAV or lentivirus vector, preferably an AAV vector comprising a capsid selected from the group consisting of: AAV1, AAV2, AAV5, AAV8, AAV2i8, AAV9, AAVrh10, AAVrh39, AAVrh43, AAVrh74, AAV-LK03, AAV2G9, AAV.PHP, AAV-Anc80, AAV3B capsids, and chimeric capsids thereof, in particular an AAV8, AAV9 or AAVrh74 capsid, such as an AAV8 or AAV9 capsid, more preferably AAV8 capsid.

In some other embodiments, the vector is a particle or vesicle, in particular lipid-based micro- or nano-vesicle or particle.

The invention relates to a cell genetically modified by a nucleic acid construct according to the invention or a vector according to the invention, preferably a liver, muscle or hematopoietic cell, more preferably liver cell.

The invention further relates to a pharmaceutical composition comprising at least an active agent selected from a nucleic acid construct according to the invention, a vector according to the invention or a cell according to according to the invention, and a pharmaceutically acceptable carrier.

The invention relates to the pharmaceutical composition according to the invention, for use in the treatment of FGF-23 related hypophosphatemic diseases, by gene therapy or cell therapy.

In some embodiments of said use, the FGF-23 related hypophosphatemic disease is a genetic disease selected from the group comprising: X-linked hypophosphatemia (XLH), Autosomal dominant hypophosphatemic rickets (ADHR), Autosomal recessive hypophosphatemic rickets 1 (ADHR1), Autosomal recessive hypophosphatemic rickets 2 (ADHR2), Osteoglophonic dysplasia, Jansen type metaphyseal chondrodysplasia, Hypophosphatemia, dental anomalies and ectopic calcification, McCune-Albright syndrome/fibrous dysplasia, and Hypophosphatemia, skin and bone lesions, or an are acquired disease selected from the group comprising: Tumor-induced osteomalacia, Hypophosphatemic osteomalacia, complications from kidney transplantation or parenteral iron therapy, chronic kidney disease and its complications such as hyperparathyroidism; preferably X-linked hypophosphatemic rickets.

### DETAILED DESCRIPTION OF THE INVENTION

### Nucleic Acid Construct

The invention provides a nucleic acid construct for gene therapy of FGF-23 related hypophosphatemic diseases.

The nucleic acid construct of the invention codes for a FGF23 fusion protein comprising at least:
(a) a signal peptide,
(b) a FGF23 C-terminal peptide which binds the FGFR/klotho complex,
(c) a cleavable linker, and
(d) a protein stabilizing moiety,
   wherein the signal peptide is at the N-terminus of the fusion protein and the FGF23 C-terminal peptide and protein stabilizing moiety are separated by the cleavable linker.

As used herein, the term Fibroblast growth factor 23 (FGF-23 or FGF23), also known as Phosphatonin or Tumor-derived hypophosphatemia-inducing factor refers to a protein encoded by the FGF23 gene in a mammalian genome. Human FGF23 has the 251 amino acid sequence UniProtKB/Swiss-Prot accession number Q9GZV9.1 or NCBI accession number NP_065689 (SEQ ID NO: 1). FGF23 is expressed as a precursor containing a N-terminal signal peptide (24 amino acids) which is cleaved to yield the mature protein (FGF23). To exert its phosphaturic activity, FGF23 requires binary FGF receptor (FGFR)-Klotho complexes. In addition, FGF23 activity is regulated by a proteolytic cleavage at the ¹⁷⁶RXXR¹⁷⁹ motif, located at the boundary between the FGF core homology domain and the 72-residue long C-terminal tail of FGF23. The proteolytic cleavage generates an inactive N-terminal fragment (Y25 to R179), the FGF core homology domain, and a C-terminal fragment (S180 to I251). FGF23 C-terminal fragment is an endogenous inhibitor or antagonist of FGF23 which competes with full-length ligand for binding to the FGFR-Klotho complex and blocks FGF23 signaling. FGF23 C-terminal fragment (180-251) was shown to antagonize phosphaturic activity of FGF23 in vivo. A smaller C-terminal fragment (FGF 180-205) was also shown to exhibit FGF23 antagonist activity (Goetz et al. PNAS, 2010, 107, 407-410). Residues 189 to 203 in mature 251-residue FGF23 are required for FGF23 activity, whereas the FGF23 amino acids 3′ to residue 203 are not necessary to initiate FGF23-dependent intracellular signaling (Garringer et al., Am. J. Physiol. Endocrinol. Metab., 2008, 295, E929-E937).

In the following description, the residues are designated by the standard one letter amino acid code and the indicated positions are determined by alignment with SEQ ID NO: 1.

“a”, “an”, and “the” include plural referents, unless the context clearly indicates otherwise. As such, the term “a” (or “an”), “one or more” or “at least one” can be used interchangeably herein; unless specified otherwise, “or” means “and/or”.

The nucleic acid construct may comprise or consist of DNA, RNA or a synthetic or semi-synthetic nucleic acid which is expressible in the individual's target cells or tissue.

The FGF23 C-terminal peptide produced by cleavage of the FGF23 fusion protein in vivo binds the FGFR/klotho complex. The mature FGF23 fusion protein (without its signal peptide) may also bind the FGFR/klotho complex. This binding inhibits FGF23 signaling through the FGFR-klotho complex. The binding activity of the FGF23 fusion protein and derived C-terminal peptide to the FGFR/klotho complex and the inhibition of FGF23 signaling through the FGFR-klotho complex may be verified by standard assays that are well-known in the art and disclosed for example in Goetz et al. PNAS, 2010, 107, 407-410. The FGF23 C-terminal peptide produced by cleavage of the FGF23 fusion protein according to the invention in vivo is a specific inhibitor or antagonist of FGFR-klotho dependent function of FGF23. The FGF23 fusion protein according to the invention may also be a specific inhibitor or antagonist of FGFR-klotho dependent function of FGF23. Due to the ability of the FGF23 C-terminal peptide, and maybe also of the fusion protein, to neutralize Klotho dependent function of FGF23, the FGF23 fusion protein of the invention is useful as therapeutics for the treatment of FGF23-related hypophosphatemic diseases.

The FGF23 C-terminal peptide comprises or consists of a sequence from any one of positions 175 to 189 to any one of positions 203 to 251 of SEQ ID NO: 1 or a sequence having at least 70%, 75%, 80%, 85%, 90%, 91%, 92%, 93%, 94%, 95%, 96%, 97%, 98%, or 99% identity with said sequence, which binds the FGFR/klotho complex. The FGF23 C-terminal peptide may comprise a sequence from position 175, 176, 177, 178, 179, 180, 181, 182, 183, 184, 185, 186, 187, 188 or 189 to position 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250 or 251 of SEQ ID NO: 1 or a sequence having at least 70%, 75%, 80%, 85%, 90%, 91%, 92%, 93%, 94%, 95%, 96%, 97%, 98%, or 99% identity with said sequence. Preferably, the FGF23 C-terminal peptide comprises or consists of a sequence from any one of positions 175 to 180 to any one of positions 205 to 251 of SEQ ID NO: 1 or a sequence having at least 70%, 75%, 80%, 85%, 90%, 91%, 92%, 93%, 94%, 95%, 96%, 97%, 98%, or 99% identity with said sequence, which binds the FGFR/klotho complex.

In some particular embodiments, the FGF23 C-terminal peptide of the invention comprises the ¹⁷⁶RXXR¹⁷⁹ motif (positions 176 to 179 of SEQ ID NO: 1). In some particular embodiments, the FGF23 C-terminal peptide of the invention terminates at position 203 of SEQ ID NO: 1. In some other particular embodiments, the FGF23 C-terminal peptide of the invention terminates at position 204 or more of SEQ ID NO: 1, such as for example at position 232 or 251 of SEQ ID NO: 1. In some preferred embodiments, the FGF23 C-terminal peptide of the invention comprises or consists of the sequence SEQ ID NO: 2 (position 175 to 251 of SEQ ID NO: 1) or a sequence having at least 70%, 75%, 80%, 85%, 90%, 91%, 92%, 93%, 94%, 95%, 96%, 97%, 98%, or 99% identity with said sequence, which binds the FGFR/klotho complex. In some embodiments, the FGF23 C-terminal peptide of the invention comprises mutations, in particular mutations which increase its binding affinity for FGFR/klotho complex.

The FGF23 C-terminal peptide according to the invention comprises or consists of a 15 to 77 amino acids C-terminal fragment of FGF23. Therefore, said FGF23 C-terminal peptide is different from the full-length FGF23 protein and does not comprise any sequence from the N-terminal region of FGF23 (positions 25 to 174 of SEQ ID NO: 1). In some embodiments, the FGF23 C-terminal peptide according to the invention comprises or consists of a C-terminal fragment of at least 20, 25, 30 or more amino acids of FGF23.

The percent amino acid sequence or nucleotide sequence identity is defined as the percent of amino acid residues or nucleotides in a Compared Sequence that are identical to the Reference Sequence after aligning the sequences and introducing gaps if necessary, to achieve the maximum sequence identity and not considering any conservative substitutions for amino acid sequences as part of the sequence identity. Sequence identity is calculated over the entire length of the Reference Sequence. Alignment for purposes of determining percent amino acid sequence identity can be achieved in various ways known to a person of skill in the art, for instance using publicly available computer software such as BLAST (Altschul et al., J. Mol. Biol., 1990, 215, 403-). When using such software, the default parameters, e.g., for gap penalty and extension penalty, are preferably used. The BLASTP program uses as default a word length (W) of 3 and an expectation (E) of 10.

The FGF23 fusion protein comprises a signal peptide at its N-terminus. Signal peptides (SP) are short peptide sequences which are present at the N-terminus of secretory and are used to target proteins for secretion. Signal peptides do not consist of a strict consensus sequence but have a three-region design consisting of a positively charged N-terminal region (N-region, 1-5 residues), a hydrophobic central region (H-region, 7-15 residues) and a neutral, polar C-terminal region (C-region, 3-5 residues). Multiple signal peptides are known in the art and publically available (see in particular, Signal Peptide Website and SPdb sequence databases; Puzzo et al., Sci. Transl. Med., 2017, 9(418): doi:10.1126). In addition, methods to select suitable SP sequences for efficient protein secretion are known in the art (see in particular, Stem et al., BMC Proc., 2011, 5 (suppl 8):013).

The signal peptide may be FGF23 endogenous or native signal peptide (SEQ ID NO: 3; positions 1 to 24 of SEQ ID NO: 1) or a heterologous signal peptide. As used herein, a heterologous signal peptide refers to a signal peptide which is different from FGF23 signal peptide, in particular human FGF23 signal peptide. Examples of heterologous signal peptides which can be used in the present invention include without limitation: alpha-1 antitrypsin (SEQ ID NO: 4); synthetic mut1 (SEQ ID NO: 5); synthetic mut3 (SEQ ID NO: 6); chymotrypsinogen B2 (CTRB2), (positions 1 to 18 of Uniprot accession number Q6GPI1 or NCBI accession number NP_001020371 or SEQ ID NO: 7) and plasma protease inhibitor C1 (positions 1 to 22 of Uniprot accession number P05155 or SEQ ID NO: 8).

In some embodiments, the signal peptide is a heterologous signal peptide, preferably the chymotrypsinogen B2 signal peptide (SEQ ID NO: 7).

The FGF23 C-terminal peptide is linked to a protein stabilizing moiety via a cleavable linker. The protein stabilizing moiety is any protein moiety which increases the half-life or duration of action of the therapeutic protein/peptide that is attached to it and is suitable for therapeutic application. Various protein stabilizing moieties that have been used to stabilize therapeutic proteins are known in the art (see for example Sven Berger, Peter Lowe & Michael Tesar (2015) Fusion protein technologies for biopharmaceuticals: Applications and challenge, mAbs, 7:3, 456-460, DOI:10.1080/19420862.2015.1019788). Examples of protein stabilizing moieties which can be used in the present invention include without limitation: serum albumin, in particular human serum albumin; immunoglobulin Fc fragment; human chorionic gonadotropin carboxy-terminal peptide (CTP); Receptor (fused to its ligand (GHR fused to GH); and latency-associated peptide of TGF-beta (linked to a cleavage site for metalloprotease).

In some embodiments, the protein stabilizing moiety is different from immunoglobulin Fc fragment.

In some embodiments, the protein stabilizing moiety is from a serum transport protein. Serum transport proteins include without limitation: the albumin family of proteins and evolutionarily related serum transport proteins such as for example albumin, alpha-fetoprotein (AFP; Beattie and Dugaiczyk, Gene 1982, 20, 415-422), afamin (AFM; Lichenstein et al., J. Biol. Chem., 1994, 269, 18149-18154) and vitamin D binding protein (DBP; Cooke and David, J. Clin. Invest., 1985, 76, 2420-2424). The serum transport protein may me from any vertebrate, including mammal, bird, fish and others. The invention encompasses functional variants such as naturally occurring polymorphic variants as well as functional fragments of serum transport proteins. A functional fragment or variant of serum transport protein refers to a variant or fragment which is capable of increasing the half-life or duration of action of the therapeutic protein/peptide that is attached to it and is suitable for therapeutic application.

In some particular embodiments, the protein stabilizing moiety is albumin, including a functional fragment or variant thereof as defined above. The albumin may be derived from any vertebrate, especially any mammal, for example human, cow, sheep, or pig. Non-mammalian albumins include, but are not limited to, hen and salmon. The albumin portion of the albumin-linked polypeptide may be from a different animal than the therapeutic polypeptide portion. In particular, the albumin fusion proteins of the invention may include naturally occurring polymorphic variants of human albumin (HA) and fragments of human albumin. The albumin portion of the albumin fusion proteins may comprise the full length of the HA sequence (NCBI accession number NP_000468), preferably comprising human serum albumin without signal peptide (positions 25-609 of NCBI accession number NP_000468 or SEQ ID NO: 9) or may include one or more fragments thereof that are capable of stabilizing or prolonging the therapeutic activity. Such fragments may be of 10 or more amino acids in length or may include about 15, 20, 25, 30, 50, 70 or more contiguous amino acids from the HA sequence or may include part or all of specific domains of HA.

In some preferred embodiments, the protein stabilizing moiety is human serum albumin (NCBI accession number NP_000468), preferably comprising human serum albumin without signal peptide (positions 25-609 of NCBI accession number NP_000468 or SEQ ID NO: 9).

The cleavable linker is any peptide linker that is cleavable in vivo. Various cleavable peptide linkers that have been used in therapeutic protein constructs are known in the art. Examples of cleavable peptide linkers which can be used in the present invention include without limitation: coagulation factors activation sequence, in particular FIX activation sequence (aa 182-200 or 182-203 of NCBI accession number NP_000124): SEQ ID NO: 10 or SEQ ID NO: 11.

In some preferred embodiments, the cleavable linker comprises or consists of the sequence SEQ ID NO: 10.

In some embodiments, the signal peptide, FGF23 C-terminal peptide, cleavable linker and protein stabilizing moiety are from the N- to C-terminus of the FGF23 fusion protein, which means that the protein stabilizing moiety is fused to the C-terminus of the FGF23 C-terminal peptide.

In some embodiments, the nucleic acid construct comprises or consists of DNA.

In some other embodiments, the nucleic acid construct) comprises or consists of RNA, in particular mRNA.

Examples of preferred nucleic acid construct of the invention include:
a nucleic acid construct coding for a FGF23 protein comprising the sequence SEQ ID NO: 12, as shown in the examples of the present application and in FIG. 1A, corresponding to the construct no 12 in Table 1,
a nucleic acid construct coding for a FGF23 protein comprising the sequence SEQ ID NO: 52, as shown in the examples of the present application, corresponding to the construct no 10 in Table 1, and
a nucleic acid construct coding for a FGF23 protein comprising a sequence having at least 70%, 75%, 80%, 85%, 90%, 91%, 92%, 93%, 94%, 95%, 96%, 97%, 98%, or 99% identity with any of sequence SEQ ID NO: 12 or 52; preferably a sequence having at least 85%, 90%, 91%, 92%, 93%, 94%, 95%, 96%, 97%, 98%, or 99% identity with any one of said sequences; more preferably a sequence having at least 95%, 96%, 97%, 98%, or 99% identity with any one of said sequences.

SEQ ID NO: 12 comprises from its N- to C-terminus: a chymotrypsinogen B2 signal peptide (SEQ ID NO: 7), a FGF23 C-terminal peptide of SEQ ID NO: 2, a cleavable linker of SEQ ID NO: 10 and human serum albumin of SEQ ID NO: 9. SEQ ID NO: 52 comprises from its N- to C-terminus: a chymotrypsinogen B2 signal peptide (SEQ ID NO: 7), a FGF23 C-terminal peptide consisting of the sequence from positions 180 to 251 of SEQ ID NO: 1, a cleavable linker of SEQ ID NO: 10 and human serum albumin of SEQ ID NO: 9.

In some embodiments, the nucleic acid construct comprises a sequence which is codon-optimized for expression in the individual that is treated by gene therapy, preferably a human individual. Appropriate softwares for codon optimization in the desired individual are well-known in the art and publically available (see for example http://www.genscript;com/cgi-bin/rare_codon_analysis; or https://eu.idtdna.com/site/account/login?returnurl=%2FCodonOpt).

In some preferred embodiments, the nucleic acid construct comprises the nucleotide sequence SEQ ID NO: 13 or SEQ ID NO: 57 which is a codon-optimized sequence for expression in human encoding the FGF23 fusion protein of SEQ ID NO: 12, the nucleotide sequence SEQ ID NO: 51 which is a codon-optimized sequence for expression in human encoding the FGF23 fusion protein of SEQ ID NO: 52, or a sequence having at least 80%, 85%, 90%, 91%, 92%, 93%, 94%, 95%, 96%, 97%, 98%, or 99% identity with any one of said sequences; preferably a sequence having at least 85%, 90%, 91%, 92%, 93%, 94%, 95%, 96%, 97%, 98%, or 99% identity with any one of said sequences; more preferably a sequence having at least 95%, 96%, 97%, 98%, or 99% identity with any one of said sequences. Said sequence is advantageously a codon-optimized sequence for expression in human.

In some embodiments, the nucleic acid construct comprises an expression cassette wherein the coding sequence is operably linked to appropriate regulatory sequences for expression of a transgene in the individual's target cells or tissue. In some particular embodiments, the target tissue is muscle or liver cells or tissue or hematopoietic cells, more particularly liver cells or tissue. Such sequences which are well-known in the art include in particular a promoter, and further regulatory sequences capable of further controlling the expression of a transgene, such as without limitation, enhancer, terminator, intron, silencer, in particular tissue-specific silencer, and microRNA.

The promoter may be a tissue-specific, ubiquitous, constitutive or inducible promoter that is functional in the individual's target cells or tissue, in particular muscle, liver or hematopoietic cells or tissue, more particularly liver cells or tissue. Examples of constitutive promoters which can be used in the present invention include without limitation: phosphoglycerate kinase promoter (PGK), elongation factor-1 alpha (EF-1 alpha) promoter including the short form of said promoter (EFS), viral promoters such as cytomegalovirus (CMV) immediate early enhancer and promoter, cytomegalovirus enhancer/chicken beta actin (CAG) promoter, SV40 early promoter and retroviral 5′ and 3′ LTR promoters including hybrid LTR promoters. Preferred ubiquitous promoter is CAG promoter. Examples of inducible promoters which can be used in the present invention include Tetracycline-regulated promoters. The promoters are advantageously human promoters, i.e., promoters from human cells or human viruses. Such promoters are well-known in the art and their sequences are available in public sequence data base.

In some particular embodiments, the promoter is a liver-specific promoter. Non-limiting examples of liver-specific promoters which can be used in the present invention include the human alpha-1 antitrypsin promoter (hAAT) (SEQ ID NO: 14), the transthyretin promoter, the albumin promoter, the thyroxine-binding globulin (TBG) promoter, the LSP promoter (comprising a thyroid hormone-binding globulin promoter sequence, two copies of an alpha1-microglobulin/bikunin enhancer sequence, and a leader sequence; Charles R. et al., Blood Coag. Fibrinol, 1997, 8: S23-S30) and others. Other useful liver-specific promoters are known in the art, for example those listed in the Liver Specific Gene Promoter Database compiled the Cold Spring Harbor Laboratory (http://rulai.cshl.edu/LSPD/). A preferred liver-specific promoter in the context of the invention is the hAAT promoter.

In other particular embodiments, the promoter is a muscle-specific promoter. Non-limiting examples of muscle-specific promoters include the muscle creatine kinase (MCK) promoter. Non-limiting examples of suitable muscle creatine kinase promoters are human muscle creatine kinase promoters and truncated murine muscle creatine kinase [(tMCK) promoters](Wang et al, Gene Therapy, 2008, 15, 1489-99); (representative GenBank Accession No. AF188002). Human muscle creatine kinase has the Gene ID No. 1158 (representative GenBank Accession No. NC_000019.9, accessed on Dec. 26, 2012). Other examples of muscle-specific promoters include: a synthetic promoter C5.12 (spC5.12, alternatively referred to herein as “C5.12”), such as the spC5.12 or the spC5.12 promoter (disclosed in Wang et al, Gene Therapy, 2008, 15, 1489-99); the MHCK7 promoter (Salva et al., Mol Ther., 2007, 15, 320-9); myosin light chain (MLC) promoters, for example MLC2 (Gene ID No. 4633; representative GenBank Accession No. NG_007554.1, accessed on Dec. 26, 2012); myosin heavy chain (MHC) promoters, for example alpha-MHC (Gene ID No. 4624; representative GenBank Accession No. NG_023444.1, accessed on Dec. 26, 2012); desmin promoters (Gene ID No. 1674; representative GenBank Accession No. NG_008043.1, accessed on Dec. 26, 2012); cardiac troponin C promoters (Gene ID No. 7134; representative GenBank Accession No. NG_008963.1, accessed on Dec. 26, 2012); troponin I promoters (Gene ID Nos. 7135, 7136, and 7137; representative GenBank Accession Nos. NG_016649.1, NG_011621.1, and NG_007866.2, accessed on Dec. 26, 2012); myoD gene family promoters (Weintraub et al., Science, 251, 761 (1991); Gene ID No. 4654; representative GenBank Accession No. NM_002478, accessed on Dec. 26, 2012); alpha actin promoters (Gene ID Nos. 58, 59, and 70; representative GenBank Accession Nos. NG_006672.1, NG_011541.1, and NG_007553.1, accessed on Dec. 26, 2012); beta actin promoters (Gene ID No. 60; representative GenBank Accession No. NG_007992.1, accessed on Dec. 26, 2012); gamma actin promoters (Gene ID No. 71 and 72; representative GenBank Accession No. NG_011433.1 and NM_001199893, accessed on Dec. 26, 2012); muscle-specific promoters residing within intron 1 of the ocular form of Pitx3 (Gene ID No. 5309) (Coulon et al; the muscle-selective promoter corresponds to residues 11219-11527 of representative GenBank Accession No. NG_008147, accessed on Dec. 26, 2012); and the promoters described in US Patent Publication US 2003/0157064, and CK6 promoters (Wang et al 2008 doi: 10.1038/gt.2008.104). In another particular embodiment, the muscle-specific promoter is the E-Syn promoter described in Wang et al., (Gene Therapy, 2008, 15, 1489-99), comprising the combination of a MCK-derived enhancer and of the spC5.12 promoter. In a particular embodiment of the invention, the muscle-specific promoter is selected in the group consisting of a spC5.12 promoter, the MHCK7 promoter, the E-syn promoter, a muscle creatine kinase myosin light chain (MLC) promoter, a myosin heavy chain (MHC) promoter, a cardiac troponin C promoter, a troponin I promoter, a myoD gene family promoter, an alpha actin promoter, an beta actin promoter, an gamma actin promoter, a muscle-specific promoter residing within intron 1 of the ocular form of Pitx3 and a CK6 promoter. In a particular embodiment, the muscle-specific promoter is selected in the group consisting of the spC5.12, desmin and MCK promoters. In a further embodiment, the muscle-specific promoter is selected in the group consisting of the spC5.12 and MCK promoters. In a particular embodiment, the muscle-specific promoter is the spC5.12 promoter.

In some other particular embodiments, the promoter is a ubiquitous promoter. Representative ubiquitous promoters include the cytomegalovirus enhancer/chicken beta actin (CAG) promoter, the cytomegalovirus enhancer/promoter (CMV) (optionally with the CMV enhancer) [see, e.g., Boshart et al, Cell, 41:521-530 (1985)], the PGK promoter, the SV40 early promoter, the retroviral Rous sarcoma virus (RSV) LTR promoter (optionally with the RSV enhancer), the dihydrofolate reductase promoter, the β-actin promoter, the phosphoglycerol kinase (PGK) promoter, and the EF1 alpha promoter.

In some other particular embodiments, the promoter is alpha-globin or beta-globin promoter. Beta-globin promoter is expressed exclusively in erythroid cells.

In yet other particular embodiments, the promoter is an endogenous promoter such as the albumin promoter or the GDE (glycogen debrancher enzyme) promoter. GDE is amylo-1,6-glucosidase 4-alpha-glucanotransferase or AGL, corresponding to human Gene ID: 178 (representative GenBank Accession No. NG_012865, accessed on 16 Sep. 2018).

In particular embodiments, the promoter is associated to an enhancer sequence, such as a cis-regulatory module (CRMs) or an artificial enhancer sequence. CRMs useful in the practice of the present invention include those described in Rincon et al., Mol Ther., 2015, 23, 43-52, Chuah et al., Mol Ther. 2014, 22, 1605-13 or Nair et al., Blood, 2014 123, 20, 3195-9. Other regulatory elements that are, in particular, able to enhance muscle-specific expression of genes, in particular expression in cardiac muscle and/or skeletal muscle, are those disclosed in WO2015110449. Particular examples of nucleic acid regulatory elements that comprise an artificial sequence include the regulatory elements that are obtained by rearranging the transcription factor binding sites (TFBS) that are present in the sequences disclosed in WO2015110449. Said rearrangement may encompass changing the order of the TFBSs and/or changing the position of one or more TFBSs relative to the other TFBSs and/or changing the copy number of one or more of the TFBSs. For example, a nucleic acid regulatory element for enhancing muscle-specific gene expression, in particular cardiac and skeletal muscle-specific gene expression, may comprise binding sites for E2A, HNH 1, NF1, C/EBP, LRF, MyoD, and SREBP; or for E2A, NF1, p53, C/EBP, LRF, and SREBP; or for E2A, HNH 1, HNF3a, HNF3b, NF1, C/EBP, LRF, MyoD, and SREBP; or E2A, HNF3a, NF1, C/EBP, LRF, MyoD, and SREBP; or for E2A, HNF3a, NF1, CEBP, LRF, MyoD, and SREBP; or for HNF4, NF1, RSRFC4, C/EBP, LRF, and MyoD, or NF1, PPAR, p53, C/EBP, LRF, and MyoD. For example, a nucleic acid regulatory element for enhancing muscle-specific gene expression, in particular skeletal muscle-specific gene expression, may also comprise binding sites for E2A, NF1, SRFC, p53, C/EBP, LRF, and MyoD; or for E2A, NF1, C/EBP, LRF, MyoD, and SREBP; or for E2A, HNF3a, C/EBP, LRF, MyoD, SEREBP, and Tal1_b; or for E2A, SRF, p53, C/EBP, LRF, MyoD, and SREBP; or for HNF4, NF1, RSRFC4, C/EBP, LRF, and SREBP; or for E2A, HNF3a, HNF3b, NF1, SRF, C/EBP, LRF, MyoD, and SREBP; or for E2A, CEBP, and MyoD. In further examples, these nucleic acid regulatory elements comprise at least two, such as 2, 3, 4, or more copies of one or more of the TFBSs recited before. Other regulatory elements that are, in particular, able to enhance liver-specific expression of genes, are those disclosed in WO2009130208. Further examples of enhancers which can be used in the present invention include the ApoE control region, in particular the human ApoE control region (or Human apolipoprotein E/C-I gene locus, hepatic control region HCR-1; Genbank accession number U32510, SEQ ID NO:15). In some more particular embodiments, an enhancer sequence such as the ApoE control region, preferably human ApoE control region, is associated to a liver-specific promoter such as those listed above, and in particular such as the hAAT promoter.

In particular embodiments, the nucleic acid construct comprises an intron, in particular an intron placed between the promoter and the coding sequence. An intron is introduced to increase mRNA stability and protein production. In addition, a modified intron designed to decrease the number of, or even totally remove, alternative open reading frames (ARFs) found in said intron can significantly improve the expression of the transgene. Furthermore, by decreasing the number of ARFs within the intron included within the construct of the invention, it is believed that the construct immunogenicity is also decreased. Preferably, ARFs are removed whose length spans over 50 bp and have a stop codon in frame with a start codon. ARFs may be removed by way of nucleotide substitution, insertion or deletion, preferably by nucleotide substitution. For example, an ATG or a GTG may be replaced by a CTG, which is not a start codon, within the sequence of the intron of interest. Examples of introns which can be used in the present invention include human beta globin b2 (or HBB2; SEQ ID NO: 16) intron, modified HBB2 intron (SEQ ID NO:17), a coagulation factor IX (FIX) intron, in particular derived from first intron (SEQ ID NO: 18) and modified intron thereof (SEQ ID NO: 19) chicken beta-globin intron (SEQ ID NO:20) and modified intron thereof (SEQ ID NO: 21), and SV40 intron. Preferred introns are modified HBB2 intron (SEQ ID NO:17 and modified FIX intron (SEQ ID NO: 19).

In particular embodiments, the nucleic acid construct further comprises a transcription termination signal (polyadenylation signal) operably linked to the coding sequence (i.e., at the 3′-end of the coding sequence). Examples of polyA which can be used in the present invention include bovine growth hormone (bGH) polyA (SEQ ID NO: 22).

In some preferred embodiments, the expression cassette comprises, in the 5′ to 3′ orientation, a liver specific promoter, preferably hAAT promoter; the coding sequence; and a polyadenylation signal such as (bGH) polyA (SEQ ID NO: 22). In some more preferred embodiments, the expression cassette further comprises one or more further regulatory elements chosen from an enhancer, preferably human ApoE control region (SEQ ID NO:15) and an intron, preferably modified HBB2 intron (SEQ ID NO:17). An example of preferred expression cassette disclosed in the examples and in FIG. 1B and comprises the sequence SEQ ID NO: 23 comprising in the 5′ to 3′ orientation: ApoE control region (SEQ ID NO: 15), alpha-1 antitrypsin promoter (hAAT) (SEQ ID NO: 14), modified HBB2 intron (SEQ ID NO:17), the coding sequence (SEQ ID NO: 13) and (bGH) polyA (SEQ ID NO: 22.). Another example of preferred expression cassette disclosed in the examples comprises in the 5′ to 3′ orientation: ApoE control region (SEQ ID NO: 15), alpha-1 antitrypsin promoter (hAAT) (SEQ ID NO: 14), modified HBB2 intron (SEQ ID NO:17), the coding sequence (SEQ ID NO: 51) and (bGH) polyA (SEQ ID NO: 22.). A more preferred expression cassette comprises the sequence SEQ ID NO: 23.

### Vector

The invention also relates to a vector comprising the nucleic acid construct as described above.

The invention may use any vector suitable for the delivery and expression of nucleic acid into individual's cells, in particular suitable for gene therapy, more particularly targeted gene therapy directed to a target tissue or cells in the individual. Such vectors that are well-known in the art include viral and non-viral vectors, wherein said vectors may be integrative or non-integrative; replicative or non-replicative. In some particular embodiments, gene therapy is directed to muscle, liver or hematopoietic cells or tissue, more particularly liver cells or tissue.

As used herein, the term “individual” or “patient” denotes a mammal. Preferably, a patient or individual according to the invention is a human. The “individual” or “patient” includes adult, children, infant and elderly.

Non-viral vector includes the various (non-viral) agents which are commonly used to either introduce or maintain nucleic acid into individual's cells. Agents which are used to introduce nucleic acid into individual's cells by various means include in particular polymer-based, particle-based, lipid-based, peptide-based delivery vehicles or combinations thereof, such as with no limitations cationic polymer, dendrimer, micelle, liposome, exosome, microparticle and nanoparticle including lipid nanoparticle (LNP); and cell penetrating peptides (CPP). CPP are in particular cationic peptides such as poly-L-Lysine (PLL), oligo-arginine, Tat peptides, Penetratin or Transportan peptides and derivatives thereof such as for example Pip. Agents which are used to maintain nucleic acid into individual's cells (either integrated into chromosome(s) or else in extrachromosomal form) include in particular naked nucleic acid vectors such as plasmids, transposons and mini-circles, and gene-editing and RNA-editing systems. Transposon includes in particular the hyperactive Sleeping Beauty (SB100X) transposon system (Mates et al. 2009). Gene-editing and RNA-editing systems may use any site-specific endonuclease such as Cas nuclease, TALEN, meganuclease, zinc finger nuclease and the like. In addition, these approaches can advantageously be combined to introduce and maintain the nucleic acid of the invention into individual's cells.

Viral vectors are by nature capable of penetrating into cells and delivering nucleic acid(s) of interest into cells, according to a process named as viral transduction.

As used herein, the term “viral vector” refers to a non-replicating, non-pathogenic virus engineered for the delivery of genetic material into cells. In viral vectors, viral genes essential for replication and virulence are replaced with an expression cassette for the transgene of interest. Thus, the viral vector genome comprises the transgene expression cassette flanked by the viral sequences required for viral vector production.

As used herein, the term “recombinant virus” refers to a virus, in particular a viral vector, produced by standard recombinant DNA technology techniques that are known in the art.

As used herein, the term “virus particle” or “viral particle” is intended to mean the extracellular form of a non-pathogenic virus, in particular a viral vector, composed of genetic material made from either DNA or RNA surrounded by a protein coat, called the capsid, and in some cases an envelope derived from portions of host cell membranes and including viral glycoproteins.

As used herein, a viral vector refers to a viral vector particle.

A preferred vector for delivering the nucleic acid (nucleic acid construct) of the invention is a viral vector, in particular suitable for gene therapy, more particularly gene therapy directed to a target tissue or cells in the individual such as muscle, liver or hematopoietic cells or tissue, more particularly liver cells or tissue. In particular, the viral vector may be derived from a non-pathogenic parvovirus such as adeno-associated virus (AAV), a retrovirus such as a gammaretrovirus, spumavirus and lentivirus, an adenovirus, a poxvirus and an herpes virus. The viral vector is preferably an integrating vector such as AAV or lentivirus vector, preferably AAV vector. Lentivirus vector may be pseudotyped with an envelope glycoprotein from another virus for targeting the cells/tissues of interest, such as muscle cells, liver cells or hematopoietic cells. In some embodiments, lentivirus is pseudotyped with syncytin as disclosed in WO 2017/182607.

The vector comprises the viral sequences required for viral vector production such as the lentiviral LTR sequences or the AAV ITR sequences flanking the expression cassette.

In particular embodiments, the vector is a particle or vesicle, in particular lipid-based micro- or nano-vesicle or particle such as liposome or lipid nanoparticle (LNP). In more particular embodiments, the nucleic acid is RNA and the vector is a particle or vesicle as described above.

In another particular embodiment, the vector is lentivirus vector, in particular pseudotyped lentivirus vector as described above.

In another particular embodiment, the vector is an AAV vector. The human parvovirus Adeno-Associated Virus (AAV) is a dependovirus that is naturally defective for replication which is able to integrate into the genome of the infected cell to establish a latent infection. The last property appears to be unique among mammalian viruses because the integration occurs at a specific site in the human genome, called AAVS1, located on chromosome 19 (19q13.3-qter). Therefore, AAV vectors have gained considerable interest as vectors for human gene therapy. Among the favorable properties of the virus are its lack of association with any human disease, its ability to infect both dividing and non-dividing cells, and the wide range of cell lines derived from different tissues that can be infected.

AAV viruses may be engineered using conventional molecular biology techniques, making it possible to optimize these particles for cell specific delivery of nucleic acid sequences, for minimizing immunogenicity, for tuning stability and particle lifetime, for efficient degradation, for accurate delivery to the nucleus.

As is known in the art, additional suitable sequences may be introduced in the nucleic acid construct of the invention for obtaining a functional viral vector. Suitable sequences include AAV ITRs. Desirable AAV fragments for assembly into vectors include the cap proteins, including the vp1, vp2, vp3 and hypervariable regions, the rep proteins, including rep 78, rep 68, rep 52, and rep 40, and the sequences encoding these proteins. These fragments may be readily utilized in a variety of vector systems and host cells. AAV-based recombinant vectors lacking the Rep protein integrate with low efficacy into the host's genome and are mainly present as stable circular episomes that can persist for years in the target cells.

In the context of the present invention, the AAV vector comprises an AAV capsid able to transduce the target cells of interest, in particular muscle, liver or hematopoietic cells or tissue, more particularly liver cells or tissue. The AAV capsid may be from one or more AAV natural or artificial serotypes.

Among the serotypes of AAVs isolated from human or non-human primates (NHP) and well characterized, human serotype 2 is the first AAV that was developed as a gene transfer vector. Other currently used AAV serotypes include AAV-1, AAV-2 variants (such as the quadruple-mutant capsid optimized AAV-2 comprising an engineered capsid with Y44+500+730F+T491V changes, disclosed in Ling et al., 2016 Jul. 18, Hum Gene Ther Methods.), -3 and AAV-3 variants (such as the AAV3-ST variant comprising an engineered AAV3 capsid with two amino acid changes, S663V+T492V, disclosed in Vercauteren et al., 2016, Mol. Ther. Vol. 24(6), p. 1042), -3B and AAV-3B variants, -4, -5, -6 and AAV-6 variants (such as the AAV6 variant comprising the triply mutated AAV6 capsid Y731F/Y705F/T492V form disclosed in Rosario et al., 2016, Mol Ther Methods Clin Dev. 3, p. 16026), -7, -8, -9, -2G9, -10 such as cy10 and -rh10, rh39, -rh43, -rh74, -dj, Anc80, LK03, AAV.PHP, AAV2i8, porcine AAV serotypes such as AAVpo4 and AAVpo6, and tyrosine, lysine and serine capsid mutants of the AAV serotypes.

Alternatively to using AAV natural serotypes, artificial AAV serotypes, i.e. with a non-naturally occurring capsid protein may be used in the context of the present invention, including, without limitation, a chimeric AAV capsid, a recombinant AAV capsid, or a “humanized” AAV capsid. Such an artificial capsid may be generated by any suitable technique, using a selected AAV sequence (e.g., a fragment of a vp1 capsid protein) in combination with heterologous sequences which may be obtained from a different selected AAV serotype, non-contiguous portions of the same AAV serotype, from a non-AAV viral source, or from a non-viral source. The modified capsid can be derived also from capsid modifications inserted by error prone PCR and/or peptide insertion (e.g. as described in Bartel et al., 2011). In addition, capsid variants may include single amino acid changes such as tyrosine mutants (e.g. as described in Zhong et al., 2008). In the context of the present invention, a “modified capsid” may be a chimeric capsid or capsid comprising one or more variant VP capsid proteins derived from one or more wild-type AAV VP capsid proteins.

In some embodiments, the AAV vector is a chimeric vector, i.e. its capsid comprises VP capsid proteins derived from at least two different AAV serotypes, or comprises at least one chimeric VP protein combining VP protein regions or domains derived from at least two AAV serotypes. Examples of such chimeric AAV vectors useful to transduce liver cells are described in Shen et al., Molecular Therapy, 2007 and in Tenney et al., Virology, 2014. For example, a chimeric AAV vector can derive from the combination of an AAV8 capsid sequence with a sequence of an AAV serotype different from the AAV8 serotype, such as any of those specifically mentioned above. In other embodiments, the capsid of the AAV vector comprises one or more variant VP capsid proteins such as those described in WO2015013313, in particular the RHM4-1, RHM15-1, RHM15-2, RHM15-3/RHM15-5, RHM15-4 and RHM15-6 capsid variants, which present a high liver tropism.

In further embodiments, the AAV vector is a pseudotyped vector, i.e. its genome and capsid are derived from AAVs of different serotypes such as the above mentioned AAV serotypes. In addition, the genome of the AAV vector may either be a single stranded or self-complementary double-stranded genome (McCarty et al., Gene Therapy, 2003). Self-complementary double-stranded AAV vectors are generated by deleting the terminal resolution site from one of the AAV terminal repeats. These modified vectors, whose replicating genome is half the length of the wild type AAV genome have the tendency to package DNA dimers.

In some embodiments, the AAV vector is suitable for gene therapy directed to a target tissue or cells in the individual, in particular muscle, liver or hematopoietic cells or tissue, more particularly liver cells or tissue. In particular embodiments the AAV vector comprises a capsid selected from the group consisting of: AAV1, AAV2, AAV2i8, AAV5, AAV8, AAV9, AAVrh10, AAVrh39, AAVrh43, AAVrh74, AAV-LK03, AAV2G9, AAV.PHP, AAV-Anc80, AAV3B capsids, and chimeric capsids thereof. In some preferred embodiments, the AAV vector comprises an AAV8, AAV9, AAVrh74 or AAV2i8 capsid, in particular an AAV8, AAV9 or AAVrh74 capsid, such as an AAV8 or AAV9 capsid, more particularly an AAV8 capsid. The genome of the AAV vector may be derived from a different serotype (pseudotyped vector) and is advantageously single-stranded.

The invention also relates to an isolated cell, in particular a cell from an individual, which is genetically modified or transformed with a nucleic acid or vector of the invention. The individual is advantageously a patient to be treated. In some embodiments, the cell is a liver cell, preferably a patient's liver cell.

As used herein “liver cell” includes primary hepatocyte such as from adult or fetal liver; hepatocyte matured in vitro, hepatocyte cell line; hepatic progenitor or pluripotent stem cell such as induced pluripotent stem cell (iPS cell), embryonic stem cells, fetal stem cell and adult stem cell.

As used herein, the term “hematopoietic cells” refers to cells produced by the differentiation of hematopoietic stem cells (HSCs or HSC). Hematopoietic cells include HSCs, multipotent and lineage-committed progenitors, precursor cells and mature cells. Mature hematopoietic cells include with no limitations, lymphocytes (B, T), NK cells, monocytes, macrophages, granulocytes, erythrocytes, platelets, plasmacytoid and myeloid dendritic cells, and microglial cells.

As used herein, the term “hematopoietic stem cells (HSCs)” refers to self-renewing cells capable of reconstituting short or long-term hematopoiesis following transplantation.

As used herein, the term “genetic modification” refers to the insertion, deletion, and/or substitution of one or more nucleotides into a genomic sequence.

As used herein muscle tissue includes in particular cardiac and skeletal muscle tissues.

As used herein, the term “muscle cells” refers to myocytes, myotubes, myoblasts, and/or satellite cells.

### Pharmaceutical Compositions and Therapeutic Uses

Another aspect of the invention is a pharmaceutical composition comprising at least an active agent selected from a nucleic acid of the invention, a vector of the invention or a cell of the invention, and a pharmaceutically acceptable carrier.

The nucleic acid, vector and derived pharmaceutical composition of the invention may be used for treating diseases by gene therapy, in particular targeted gene therapy directed to muscle, liver or hematopoietic cells or tissue, more particularly liver cells or tissue. The cell and derived pharmaceutical composition of the invention may be used for treating diseases by cell therapy, in particular cell therapy directed to muscle, liver or hematopoietic cell, preferably liver-directed cell therapy.

As used herein “Gene therapy” refers to a treatment of an individual which involves delivery of nucleic acid of interest into an individual's cells for the purpose of treating a disease. Delivery of the nucleic acid is generally achieved using a delivery vehicle, also known as a vector. Viral and non-viral vectors may be employed to deliver a gene to a patient's cells.

As used herein “Cell therapy” refers to a process wherein cells modified by a nucleic or vector of the invention are delivered to the individual in need thereof by any appropriate mean such as for example by intravenous injection (infusion), or injection in the tissue of interest (implantation or transplantation). In particular embodiments, cell therapy comprises collecting cells from the individual, modifying the individual's cells with the nucleic acid or vector of the invention, and administering the modified cells back to the patient. As used herein “cell” refers to isolated cell, natural or artificial cellular aggregate, bioartificial cellular scaffold and bioartificial organ or tissue.

A “pharmaceutically acceptable carrier” refers to a vehicle in which the therapeutic is administered and which does not produce an adverse, allergic or other untoward reaction when administered to a mammal, especially a human, as appropriate. A pharmaceutically acceptable carrier refers to a non-toxic solid or liquid filler, diluent, adjuvant, excipient, encapsulating material or formulation auxiliary of any type. The pharmaceutical composition which is formulated according to standard procedures can take the form of solutions, suspensions, emulsions, tablets, pills, capsules, powders, sustained-release formulations, implants, and others.

The pharmaceutical composition of the invention comprises a therapeutically effective amount of the nucleic acid, vector or cell therapeutic, preferably in purified form, together with a suitable amount of carrier so as to provide the form for proper administration to the subject.

In the context of the invention, a therapeutically effective amount refers to a dose sufficient for reversing, alleviating or inhibiting the progress of the disorder or condition to which such term applies, or reversing, alleviating or inhibiting the progress of one or more symptoms of the disorder or condition to which such term applies.

The effective dose is determined and adjusted depending on factors such as the composition used, the route of administration, the physical characteristics of the individual under consideration such as sex, age and weight, concurrent medication, and other factors, that those skilled in the medical arts will recognize. The effective dose can be determined by standard clinical techniques. In addition, in vivo and/or in vitro assays may optionally be employed to help predict optimal dosage ranges. In case of a treatment comprising administering a viral vector, such as an AAV vector, to the subject, typical doses of the vector are of at least 1×10⁸ vector genomes per kilogram body weight (vg/kg), such as at least 1×10⁹ vg/kg, at least 1×10¹⁰ vg/kg, at least 1×10¹¹ vg/kg, at least 1×10¹² vg/kg at least 1×10¹³ vg/kg, at least 1×10¹⁴ vg/kg.

In some embodiments, the pharmaceutical composition contains vehicles, which are pharmaceutically acceptable for a formulation capable of being injected, in particular to human individuals. These include in particular sterile isotonic aqueous solutions or suspensions, such as saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The solution or suspension may comprise additives which are compatible with nucleic acids and viral vectors and do not prevent nucleic acids or viral vector particle entry into target cells. In all cases, the form must be sterile and must be fluid to the extent that easy syringe ability exists. It must be stable under the conditions of manufacture and storage and must be preserved against the contaminating action of microorganisms, such as bacteria and fungi. An example of an appropriate solution is a buffer, such as phosphate buffered saline (PBS) or Ringer lactate.

In some particular embodiments, the nucleic acid, vector or cell of the invention is formulated in a composition comprising phosphate-buffered saline and supplemented with 0.25% human serum albumin. In other particular embodiments, the nucleic acid, vector or cell of the invention is formulated in a composition comprising ringer lactate and a non-ionic surfactant, such as pluronic F68 at a final concentration of 0.01-0.0001%, such as at a concentration of 0.001%, by weight of the total composition. The formulation may further comprise serum albumin, in particular human serum albumin, such as human serum albumin at 0.25%. Other appropriate formulations for either storage or administration are known in the art, in particular from WO 2005/118792 or Allay et al., 2011, Hum. Gene Ther, 2011 May; 22(5):595-604.

In some other embodiments, the pharmaceutical composition is formulated as an implant. The implant may be of a porous, nonporous, or gelatinous material, including membranes, such as sialastic membranes, or fibers. The implant is useful to administer the pharmaceutical compositions of the invention locally to the area in need of treatment, i.e. the liver.

In yet other embodiments, the pharmaceutical composition is formulated as a controlled release system.

The pharmaceutical composition may also comprise an additional therapeutic agent, in particular an agent useful for the treatment of FGF-23 related diseases, in particular FGF-23 related hypophosphatemic diseases, such as in particular calcitriol.

Another aspect of the invention relates to a nucleic acid, vector, cell, pharmaceutical composition of the invention for use as a medicament.

A further aspect of the invention relates to a nucleic acid, vector, cell, pharmaceutical composition of the invention for use in the treatment of a FGF-23 related disease, in particular FGF-23 related hypophosphatemic diseases, by gene therapy or cell therapy as described above, preferably liver-directed gene therapy or cell therapy.

Yet another aspect of the invention relates to the use of a nucleic acid, vector, cell, pharmaceutical composition of the invention in the manufacture of a medicament for the treatment of a FGF-23 related disease, in particular FGF-23 related hypophosphatemic diseases, by gene therapy or cell therapy as described above, preferably liver-directed gene therapy or cell therapy.

The nucleic acid construct, vector, composition according to the invention are used for treating diseases that can be treated by inhibition of FGF23-FGF receptor (FGFR)-Klotho complex formation, such as diseases caused by excessive action of FGF23, in particular mediated by interaction of FGF23 with FGFR/klotho complex.

The diseases that can be treated are in particular FGF-23 related hypophosphatemic diseases. These diseases can be diagnosed by high FGF23 levels in the presence of hypophosphatemia because FGF23 levels are low in hypophosphatemic patients from other causes.

Examples of FGF-23 related hypophosphatemic diseases that can be treated include genetic diseases such as X-linked hypophosphatemia (XLH) caused by mutations in the PHEX gene; Autosomal dominant hypophosphatemic rickets (ADHR) caused by mutations in the FGF23 gene; Autosomal recessive hypophosphatemic rickets 1 (ADHR1) caused by mutations in the DMP1 gene; Autosomal recessive hypophosphatemic rickets 2 (ADHR2) caused by mutations in the ENPP1 gene; Osteoglophonic dysplasia caused by mutations in the FRFR1 gene; Jansen type metaphyseal chondrodysplasia caused by mutations in the PTH1R gene; Hypophosphatemia, dental anomalies and ectopic calcification caused by mutations in the FAM20C gene; McCune-Albright syndrome/fibrous dysplasia caused by mutations in the GNAS1 gene; Hypophosphatemia, skin and bone lesions caused by mutations in the HRAS or NRAS gene.

Other examples of FGF-23 related hypophosphatemic diseases that can be treated are acquired diseases such as Tumor-induced osteomalacia (TIO), Hypophosphatemic osteomalacia by saccharated ferric oxide or iron polymaltose, complications from kidney transplantation or parenteral iron therapy, chronic kidney disease and its complications such as hyperparathyroidism.

In some preferred embodiments, said FGF-23 related hypophosphatemic disease is a genetic disease, preferably XLH. Said disease is preferably treated by targeted gene therapy, in particular directed to muscle, liver or hematopoietic cells or tissue, more particularly liver cells or tissue, or by cell therapy, more preferably using an AAV or lentivirus vector, in particular AAV8 vector.

Another aspect of the invention relates to a method of treating FGF-23 related hypophosphatemic disease as describe above, comprising: administering to a patient a therapeutically effective amount of the nucleic acid, vector, cell or pharmaceutical composition as described above.

In the context of the invention, the term “treating” or “treatment”, as used herein, means reversing, alleviating or inhibiting the progress of the disorder or condition to which such term applies, or reversing, alleviating or inhibiting the progress of one or more symptoms of the disorder or condition to which such term applies.

The nucleic acid, vector, cell or pharmaceutical composition of the invention are generally administered according to known procedures, at dosages and for periods of time effective to induce a therapeutic effect in the patient. The nucleic acid of the invention, whether vectorized or not, may be administered by any convenient route, such as in a non-limiting manner by infusion or bolus injection, by absorption through epithelial or mucocutaneous linings (e.g., oral mucosa, rectal and intestinal mucosa, etc.). Administration can be systemic or local; systemic includes parenteral and oral, and local includes local and loco-regional. The parenteral administration is advantageously by injection or perfusion, such as subcutaneous (SC), intramuscular (IM), intravascular such as intravenous (IV), intraarterial, intraperitoneal (IP), intradermal (ID), intranasal, epidural or else. In addition, it may be desirable to introduce the pharmaceutical compositions of the invention into the liver of the subject by any suitable route.

The nucleic acid, vector, cell or pharmaceutical composition of the invention and may be used in combination with other biologically active agents, wherein the combined use is by simultaneous, separate or sequential administration.

The practice of the present invention will employ, unless otherwise indicated, conventional techniques, which are within the skill of the art. Such techniques are explained fully in the literature.

The invention will now be exemplified with the following examples, which are not limitative, with reference to the attached drawings in which:

### FIGURE LEGENDS

FIG. 1. Schematic representation of nucleic acid construct and AAV expression vector for truncated FGF23 fusion protein according to the invention.

A. Nucleic acid construct for truncated FGF23 fusion protein according to the invention. The C-terminal amino acids of the human FGF protein (aa 175-251, cFGF) were fused with human serum albumin without signal peptide (aa 25-609) through a cleavable linker derived from amino acids 182-200 of the human coagulation factor IX (hFIX). Finally, a signal peptide derived from the first 18 amino acids of chymotrypsinogen B2 was fused at the N-terminal of the construct (CTRB2).

B. AAV expression vector for truncated FGF23 fusion protein according to the invention. The nucleic acid construct was inserted in an AAV expression vector comprising a liver-specific promoter (Apoliprotein E (ApoE) enhancer—human alpha1 antitrypsin (hAAT) promoter), modified HBB2 intron and bovine growth hormone (bGH) polyadenylation signal.

FIG. 2. Stabilization of the FGF23 c-terminal part of by albumin fusion in vitro. Human liver hepatoma cells (Huh-7) were transfected with plasmids expressing different versions of human FGF23 as described in Table 1. Forty-eight hours after transfection, the medium was harvested and analyzed by Western blot with an anti-FGF23 antibody. (A) Comparison of all the constructs showing the relative expression of the different constructs and the higher expression levels achieved with the fusion proteins of FGF23-C and albumin moieties. The positions of FGF23, FGF23-C or the fusion proteins of FGF23-C with human albumin (FGF-23-Albumin) are indicated on the left. (B) Comparison of the expression levels achieved after transfection in triplicate of plasmids expressing the FGF23-C Albumin fusion proteins in Huh-7 cells.

FIG. 3. Stabilization of the c-terminal part of FGF23 by albumin fusion in vivo. Wild-type C57BL6/J mice were injected with 1×10¹² vg/mouse of AAV8 vectors expressing the codon optimized version of the c-terminal part of FGF23 (coFGF23-C, amino acids 175-251) containing the RRHTR motif (only constructs 7, 9 and 12), the sp7 signal peptide and fused (constructs 9, 11 and 12) or not (construct 7) with albumin moiety. Construct 12 included a linker composed of amino acids 182-200 of the human coagulation factor IX whereas construct 9 and 11 had no linker. One month after vector injection, mice were sacrificed and plasma and liver collected to measure phosphatemia and the levels of expression achieved with the different AAV vectors. (A) Phosphatemia measured in blood. (B) Vector genome copy number (VGCN) per diploid genome measured in liver. (C) Western blot analysis performed in plasma with an anti-FGF23 antibody. (D) The histogram showed the relative intensity of the bands depicted in panel C. Statistical analysis was performed by t test (* P<0.05 vs. PBS-treated mice, n=4-5 per group).

FIG. 4. AAV-mediated rescue of the growth delay in Hypduk mice One-month old Hypduk mice were treated either with PBS (KO, PBS) or with 1×10¹² vg/mouse of an AAV vector expressing the FGF23-C fused with sp7 and albumin through the hFIX linker and containing the RRHTR motif (KO, AAV). PBS-treated wild-type mice were used as controls (WT, PBS). A to C. Mice were measured one, two and three months after vector injection. In (A) weight, (B) tail length and (C) body size gains over three months were shown. Statistical analyses were performed by ANOVA (* P<0.05 vs. KO, PBS; #P<0.05 vs. WT, PBS; n=9-11 per group).

FIG. 5. AAV-mediated rescue of hypophosphatemia in Hypduk mice One month-old Hypduk mice were treated either with PBS (KO, PBS) or with 1×10¹² vg/mouse of an AAV vector expressing the FGF23-C fused with sp7 and albumin through the hFIX linker and containing the RRHTR motif (KO, AAV). PBS-treated wild-type mice were used as controls (WT, PBS). Phosphate levels in blood were measured three months after vector injection. Statistical analyses were performed by ANOVA (*=P<0.05 as indicated, NS not significant).

FIG. 6. AAV-mediated rescue of muscle function in Hypduk mice One month-old Hypduk mice were treated either with PBS (KO, PBS) or with 1×10¹² vg/mouse of an AAV vector expressing the FGF23-C fused with sp7 and albumin through the hFIX linker and containing the RRHTR motif (KO, AAV). PBS-treated wild-type mice were used as controls (WT, PBS). Inverted grid performance measured three months after vector injection. Statistical analyses were performed by ANOVA (*=P<0.05 as indicated, NS not significant).

### EXAMPLES

### Material and Methods

### Construction of Nucleic Acids Encoding Truncated FGF23

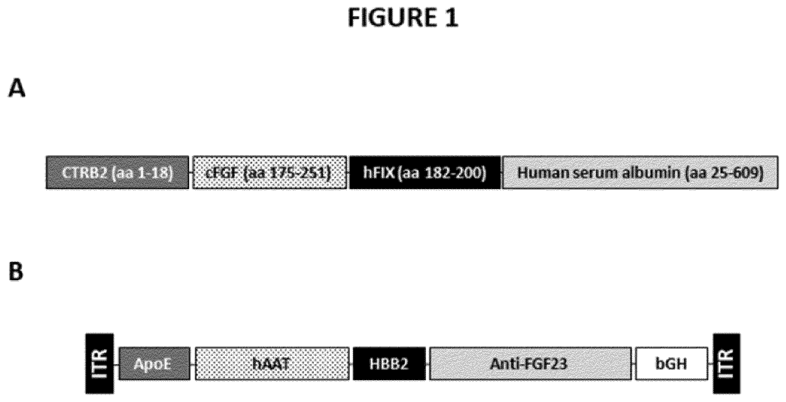

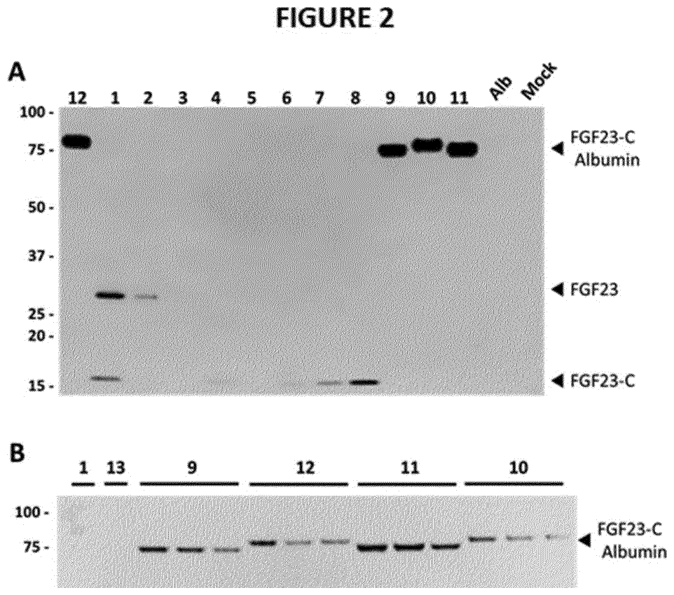

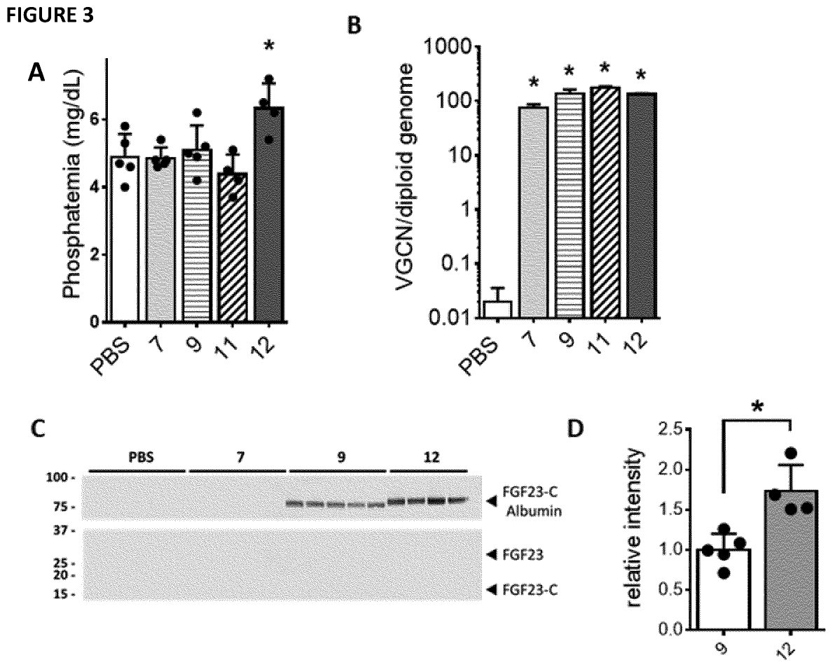

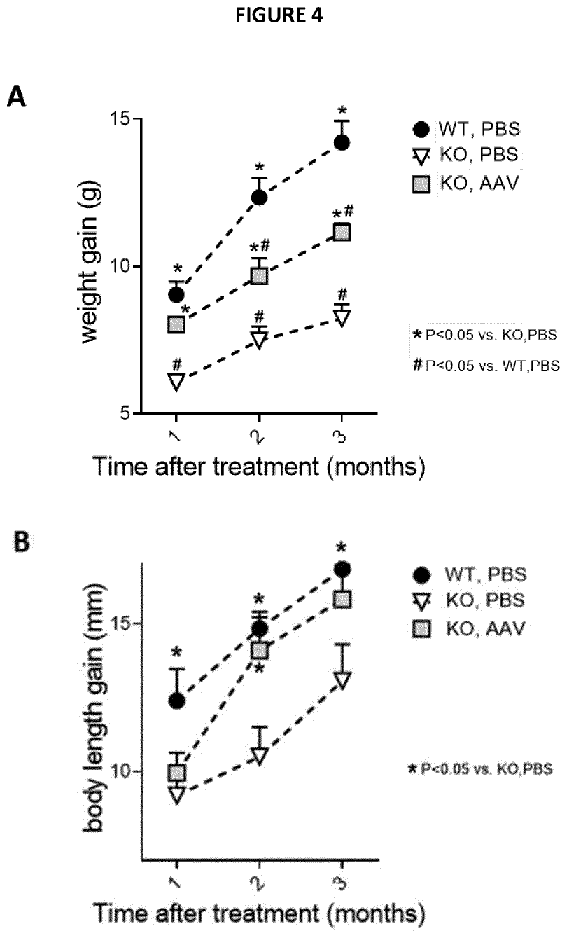

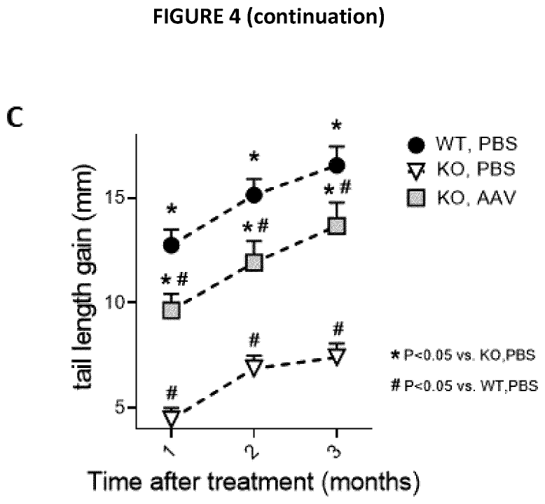

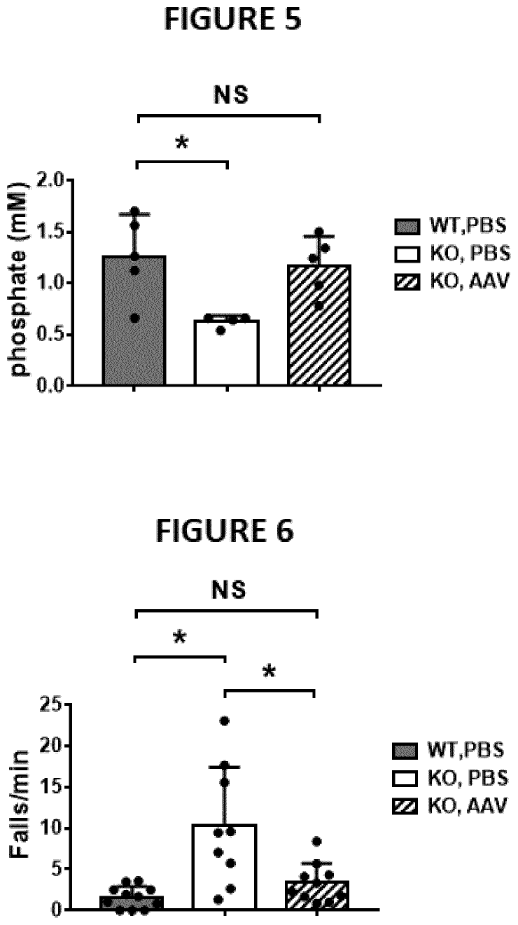

Nucleic acid constructs encoding truncated FGF23 were generated by fusing amino acids 175-251 of human FGF23 protein (aa 175-251 of NP_065689; SEQ ID NO: 2) comprising the RXXR motif (amino acids 175-179 of human FGF23 protein) preceded by R175 (RRHTR motif; amino acids 175-179 of human FGF23 protein) or amino acids 180-251 of the human FGF protein of SEQ ID NO: 1 (not comprising said RXXR motif), with human serum albumin without signal peptide (aa 25-609 of NP_000468 or SEQ ID NO: 9) through a cleavable linker derived from human coagulation factor IX (cFIX; aa 182-200 of NP_000124; SEQ ID NO: 10). Finally, a signal peptide derived from the first 18 amino acids of chymotrypsinogen B2 (sp7; aa 1-18 of NP_001020371 or SEQ ID NO: 7) was inserted at the N-terminal of the constructs to mediate an efficient secretion (SEQ ID NO: 12 (FIG. 1A); SEQ ID NO: 52). These sequences were codon optimized by a commercial algorithm. The resulting sequences are SEQ ID NO: 57 which comprises the CDS of SEQ ID NO: 13 which encodes the fusion protein of SEQ ID NO: 12, flanked in 5′ with MluI/Kozak (construct no 12 in Table 1); and SEQ ID NO: 51 which comprises the CDS which encodes the fusion protein of SEQ ID NO: 52, flanked in 5′ with MluI/Kozak (construct no 10 in Table 1). The codon optimized sequences were cloned into a transgene expression cassette optimized for liver expression (FIG. 1B).

Other constructs were generated for comparison (Table 1). Nucleic acid constructs encoding native human FGF23 (SEQ ID NO: 1) were generated by cloning the wild-type or the codon optimized human FGF23 sequence in the transgene expression cassette optimized for liver expression (FIG. 1B)

Truncated C-terminal FGF23 constructs (FGF23-C) were generated by fusing the FGF23-C containing or not the RXXR motif (amino acids 175-251 or 180-251 of human FGF23 protein) with the native FGF23 or sp7 signal peptides. These sequences were codon optimized by a commercial algorithm and cloned into the transgene expression cassette optimized for liver expression.

Chimeric proteins were generated, either by fusion of the FGF23-C not containing the RXXR motif with human serum albumin (without signal peptide), without the cleavable linker derived from human coagulation factor IX, or by fusion of the FGF23-C containing the RXXR motif with human serum albumin (without signal peptide) without the cleavable linker derived from human coagulation factor IX. The signal peptide derived from the first 18 amino acids of chymotrypsinogen B2 (sp7) was inserted at the N-terminal of the construct to mediate an efficient secretion. These sequences were codon optimized by a commercial algorithm and cloned into the transgene expression cassette optimized for liver expression (FIG. 1B).

### AAV Vector Production

AAV vectors were produced using an adenovirus-free transient transfection method (Matsushita et al., Gene Therapy, 1998, 5, 938-945) and purified as described earlier (Ayuso et al., Gene Therapy, 2010, 17, 503-510). Titers of the AAV vector stocks were determined using a quantitative real-time PCR (qPCR) and confirmed by SDS-PAGE followed by SYPRO® Ruby protein gel stain and band densitometry.

### In Vitro Studies

Huh-7 at 70-80% confluence were transfected by lipofectamine with the constructs indicated in Table 1. Two days after transfection, the medium was harvested and the levels of FGF23 were analyzed by Western blot.

### Western Blot

Huh-7 medium after transfection and serum from mice injected with the different AAV vectors were loaded onto a 4-15% gradient polyacrylamide gel to perform SDS-PAGE. After transfer on nitrocellulose, the membrane was blocked and incubated with an anti-FGF23 antibody. The membrane was incubated with the appropriate secondary antibody, and visualized by Odyssey imaging system.

### Mouse Studies

AAV vectors were administered intravenously via the tail vein to six week-old C57BL6/J mice. One month after vector injection, the levels of FGF23-C in circulation were analysed by Western blot. Livers were harvested and vector genome copy numbers were measured by qPCR

AAV vectors were administered intravenously via the tail vein to one month-old male Hypduk mice. PBS injected, wild-type and Hypduk littermates were used as controls. Mice were weighted and measured monthly for 3 months after vector injection, to evaluate the correction of the disease at macroscopic level. The measurement of blood phosphatemia and the functional evaluation of muscle strength were performed three months after vector injection.

### Phosphatemia

To measure circulating phosphate levels, mice were bled three months after vector injection. After centrifugation at 10000×g for 10 minutes, the inorganic phosphate content in serum was measured using standard commercial kit.

### Inverted Grid Test

Mice were placed on a grid and allowed to accommodate for 3 to 5 seconds before the grid is inverted and held at least 35 cm over a mouse cage containing 5 to 7 cm of soft bedding.

The number of falls was measured over a period of three minutes and reported as falls per minute.

### Results

The C-terminal portion of the human FGF23 (FGF23-C) competes with native FGF23 and reduces the intracellular signal transmission after receptor binding.

One important limitation in the use of FGF23-C as a therapy for XLH is that this peptide is very unstable in the circulation. A gene therapy approach was developed to secrete this peptide from the liver and improve its stability. Amino acids 175-251 of the human FGF protein (SEQ ID NO: 2) comprising the RXXR motif preceded by R175 (RRHTR motif; amino acids 175-179 of human FGF23 protein) or amino acids 180-251 of the human FGF protein of SEQ ID NO: 1 (not comprising said RXXR motif) were fused with human serum albumin without signal peptide (SEQ ID NO: 9) through a cleavable linker derived from human coagulation factor IX (SEQ ID NO: 10). Finally, a signal peptide derived from the first 18 amino acids of chymotrypsinogen B2 (SEQ ID NO: 7) was inserted at the N-terminal of the constructs to mediate an efficient secretion (SEQ ID NO: 12 (FIG. 1A); SEQ ID NO: 52). These sequences were codon optimized by a commercial algorithm. The resulting sequences are SEQ ID NO: 57 which comprises the CDS of SEQ ID NO: 13 which encodes the fusion protein of SEQ ID NO: 12, flanked in 5′ with MluI/Kozak (construct no 12 in Table 1); and SEQ ID NO: 51 which comprises the CDS which encodes the fusion protein of SEQ ID NO: 52, flanked in 5′ with MluI/Kozak (construct no 10 in Table 1). The codon optimized sequences were cloned into a transgene expression cassette optimized for liver expression (FIG. 1B). The transgene expression cassette comprising SEQ ID NO: 13 included in SEQ ID NO: 57 had the sequence SEQ ID NO: 23. The AAV transfer vector comprising the expression cassette of SEQ ID NO: 23 flanked by AAV ITRs had the sequence SEQ ID NO: 24.

To confirm the low stability of FGF23-C and to test strategies to improve its secretion and stability, the inventors transfected human liver hepatoma cells with different constructs expressing both native FGF23 and FGF23-C under the transcriptional control of the hAAT promoter (Table 1 and FIG. 1).

TABLE 1
Table 1. Description of the constructs tested.

| No | Signal | RXXR | FGF23 | cFIX | Albumin |
|---|---|---|---|---|---|
| 1 | spFGF23wt | + (wt) | wtFGF23 | − | − |
| 2 | spFGF23wt | + (co) | coFGF23 | − | − |
| 3 | spFGF23wt | + (co) | wtFGF23-C | − | − |
| 4 | spFGF23wt | − | wtFGF23-C | − | − |
| 5 | spFGF23co | + (co) | coFGF23-C | − | − |
| 6 | spFGF23co | − | coFGF23-C | − | − |
| 7 | sp7co | + (co) | coFGF23-C | − | − |
| 8 | sp7co | − | coFGF23-C | − | − |
| 9 | sp7co | + (co) | coFGF23-C | − | + |
| 10 | sp7co | − | coFGF23-C | + | + |
| 11 | sp7co | − | coFGF23-C | − | + |
| 12 | sp7co | + (co) | coFGF23-C | + | + |
| 13 | sp7co | − | — | − | + |

FGF23, fibroblast growth factor 23; FGF23-C, c-terminal part of FGF23 (amino acid 175-251); spFGF23, native FGF23 signal peptide, sp7, chymotrypsinogen B2 signal peptide; RXXRR, amino acids 176-179 of FGF23-C; wt, native FGF23 sequence; co, codon-optimized; cFIX, amino acids 182-200 of the human coagulation factor IX; Albumin, amino acids 25-609 of the human serum albumin sequence.

Construct no 1 (nucleotide (nt) sequence (SEQ ID NO: 25); corresponding amino acid (aa) sequence SEQ ID NO: 26, 1 or 27).
Construct no 2 (nucleotide (nt) sequence (SEQ ID NO: 28); corresponding amino acid (aa) sequence SEQ ID NO: 29, 1, 26 or 27).
Construct no 3 (nucleotide (nt) sequence (SEQ ID NO: 30); corresponding amino acid (aa) sequence SEQ ID NO: 31 or 32).
Construct no 4 (nucleotide (nt) sequence (SEQ ID NO: 33); corresponding amino acid (aa) sequence SEQ ID NO: 34 or 35).
Construct no 5 (nucleotide (nt) sequence (SEQ ID NO: 36); corresponding amino acid (aa) sequence SEQ ID NO: 37 or 38).
Construct no 6 (nucleotide (nt) sequence (SEQ ID NO: 39); corresponding amino acid (aa) sequence SEQ ID NO: 40 or 41).
Construct no 7 (nucleotide (nt) sequence (SEQ ID NO: 42); corresponding amino acid (aa) sequence SEQ ID NO: 43 or 44).
Construct no 8 (nucleotide (nt) sequence (SEQ ID NO: 45); corresponding amino acid (aa) sequence SEQ ID NO: 46 or 47).
Construct no 9 (nucleotide (nt) sequence (SEQ ID NO: 48); corresponding amino acid (aa) sequence SEQ ID NO: 49 or 50).
Construct no 10 (nucleotide (nt) sequence (SEQ ID NO: 51); corresponding amino acid (aa) sequence SEQ ID NO: 52 which is identical to SEQ ID NO: 53).
Construct no 11 (nucleotide (nt) sequence (SEQ ID NO: 54); corresponding amino acid (aa) sequence SEQ ID NO: 55 or 56).
Construct no 12 (nucleotide (nt) sequence (SEQ ID NO: 57); corresponding amino acid (aa) sequence SEQ ID NO: 58 which is identical to SEQ ID NO: 12).

Wild-type and codon-optimized versions of FGF-23 and FGF23-C genes were tested together with versions fused with the native FGF23 and the heterologous signal peptide of chymotrypsinogen B2 (SEQ ID NO: 7, sp7). In the different sequences tested, the RRHTR motif, present in human FGF23 and responsible for its cleavage into the FGF23 N-terminal and C-terminal peptides was either included or not.

Native FGF23 was efficiently produced and secreted in the medium of transfected Huh-7 cells as well as its codon optimized version (constructs no 1 and 2, FIG. 2A). None of the FGF23-C variants was produced in the medium with the exception of the sp7-fused FGF23-C without the RHTR motif (construct no 8, FIG. 2A). Interestingly, fusion with albumin moiety (SEQ ID NO: 9) enhanced the secretion of FGF23-C regardless of the composition of the chimeric protein (constructs no 9-12, FIG. 2A). The fusion of FGF23-C with albumin in the absence of the RHTR motif and the cleavable linker derived from human coagulation factor IX (construct no 11) led to higher levels of secretion in vitro when compared to other chimeric proteins containing the albumin moiety (constructs no 9, no 10 and no 12, FIG. 2B).

AAV8 vectors expressing constructs no 7, no 9, no 11 and no 12 were prepared: i) FGF23-C fused with sp7 and containing the RRHTR motif (construct no 7), ii) FGF23-C fused with sp7 and albumin and containing the RRHTR motif (construct no 9), iii) FGF23-C fused with sp7 and albumin without the RRHTR motif (construct no 11), and iv) FGF23-C fused with sp7 and albumin through the hFIX linker and containing the RRHTR motif (construct no 12, Table 1 and FIG. 1A). AAV8 vectors were produced and injected in six week-old C57BL6/J mice at the dose of 1×10¹² vg/mouse. One month post vector injection, mice were bled to measure the phosphatemia and the levels of FGF23-C by Western blot. Importantly, increased phosphorus levels in blood were observed only in mice injected with an AAV8 vector expressing construct no 12 (FIG. 3A), despite similar levels of liver transduction achieved with the other constructs (FIG. 3B) and higher expression levels achieved in vitro with construct no 11.

Bands corresponding to the molecular weight of FGF23-C-albumin chimeric proteins were revealed only in mice injected with AAV8 vectors expressing constructs no 9 and no 12 specifically in the liver. Conversely, no bands with a size compatible with FGF23-C were detectable in mice injected with AAV8 vectors expressing construct no 7 i.e. the FGF23-C fused with sp7 confirming the instability of the FGF23-C in vivo (FIG. 3C). Importantly, mice injected with construct no 12 comprising a cleavable linker (hFIX; SEQ ID NO: 10) showed significantly higher levels of circulating FGF-23-C fusion protein when compared with construct no 9 (without linker; FIG. 3D) despite similar liver transduction as demonstrated by the vector genome copy number per cell (FIG. 3B) and higher expression levels achieved in vitro with construct no 11. These data indicate that the C-terminal part of FGF23 (FGF23-C) can be expressed by the liver only as fusion with albumin moiety. They also suggest that the composition of the fusion protein influence its stability and function in circulation that are increased when FGF23-C is fused with albumin moiety through a cleavable linker, in particular derived from human coagulation factor IX (SEQ ID NO: 10).

To verify if the chimeric protein composed of FGF23-C and fused with sp7 and albumin through a hFIX-derived linker according to the invention (construct no 12, Table 1 and FIG. 1A) was active in vivo, the AAV8 vector expressing this construct was injected in one month-old Hypduk mice at the dose of 1×10¹² vg/mouse (KO, AAV). PBS-injected Hypduk (KO, PBS) and wild-type littermates (WT, PBS) were used as controls. Phenotype correction was evaluated by measuring body weight and body and tail length monthly starting one month after vector injection (FIG. 4). In general, PBS-treated Hypduk mice had lower weight and body and tail length (FIG. 4A-C) when compared to wild-type animals. Hypduk mice treated with AAV8 vectors expressing the FGF23-C chimeric protein according to the invention showed weight and body and tail length gains over a 3 months period that were higher than those of PBS-treated Hypduk mice and similar to those observed in wild-type animals (FIG. 4A-C). Although a complete rescue of body length gain was observed in AAV-treated animals (FIG. 4B), weight and tail length were only partially rescued (FIG. 4A, C). Three months after treatment, the levels of phosphate in serum were measured. In AAV-treated Hypduk mice, the phosphate levels were significantly different from those measured in PBS-treated Hypduk mice and undistinguishable from phosphate levels in wild-type animals (FIG. 5). The impairment in bones growth and calcification of the tendons may result in decreased motor function and muscle strength. Functional evaluation of the phenotype in Hypduk mice was performed by inverted grid test. We observed an increased number of fall per minute in Hypduk mice compared to wild-type that was totally rescued by AAV gene therapy (FIG. 6)

1-18. (canceled)

19. A nucleic acid construct for gene therapy of FGF23 related hypophosphatemic diseases, which codes for a FGF23 fusion protein comprising:
(a) a signal peptide,
(b) a FGF23 C-terminal peptide which binds the FGFR/klotho complex,
(c) a cleavable linker, and
(d) a protein stabilizing moiety,
wherein the signal peptide is at the N-terminus of the fusion protein and the FGF23 C-terminal peptide and protein stabilizing moiety are separated by the cleavable linker.

20. The nucleic acid construct according to claim 19, wherein the FGF23 C-terminal peptide comprises a sequence from any one of positions 175 to 189 to any one of positions 203 to 251 of SEQ ID NO: 1 or a sequence having at least 70%, 75%, 80%, 85%, 90%, 91%, 92%, 93%, 94%, 95%, 96%, 97%, 98%, or 99% sequence identity with said sequence.

21. The nucleic acid construct according to claim 19, wherein the FGF23 C-terminal peptide comprises the RXXR motif in positions 176 to 179 of SEQ ID NO: 1.

22. The nucleic acid construct according to claim 19, wherein the FGF23 C-terminal peptide comprises the sequence SEQ ID NO: 2 or a sequence having at least 70%, 75%, 80%, 85%, 90%, 91%, 92%, 93%, 94%, 95%, 96%, 97%, 98%, or 99% sequence identity with said sequence.

23. The nucleic acid construct according to claim 19, wherein the signal peptide comprises a sequence selected from the group consisting of SEQ ID NO: 3 to 8; preferably SEQ ID NO: 7, and/or the cleavable linker comprises the sequence SEQ ID NO: 10.

24. The nucleic acid construct according to claim 19, wherein the protein stabilizing moiety is human serum albumin, in particular comprising the sequence SEQ ID NO: 9.

25. The nucleic acid construct according to claim 19, which codes for a FGF23 protein comprising the sequence SEQ ID NO: 12 or 52 or a sequence having at least 85%, 90%, 91%, 92%, 93%, 94%, 95%, 96%, 97%, 98%, or 99% sequence identity with any one of said sequences.

26. The nucleic acid construct according to claim 19 which is codon optimized for expression in a human.

27. The nucleic acid construct according to claim 25, which comprises the sequence SEQ ID NO: 13, 51 or 57 or a sequence having at least 80%, 85%, 90%, 91%, 92%, 93%, 94%, 95%, 96%, 97%, 98%, or 99% identity with any one of said sequences.

28. The nucleic acid construct according to claim 19, which comprises an expression cassette wherein the coding sequence is operably linked to at least a promoter that is functional in muscle, liver or hematopoietic cells or tissue, in particular a liver-specific promoter such as human alpha-1 antitrypsin promoter.

29. The nucleic acid construct according to claim 28, which further comprises one or more control elements selected from the group consisting of: an enhancer associated to the promoter, preferably human ApoE control region; an intron placed between the promoter and the coding sequence such as a modified HBB2 intron of SEQ ID NO: 17 or a modified FIX intron of SEQ ID NO: 19; and a transcription termination signal such as bovine growth hormone polyadenylation signal.

30. The nucleic acid construct according to claim 19, which is DNA or RNA.

31. A vector for gene therapy comprising the nucleic acid construct of claim 19.

32. The vector according to claim 31, which is a viral vector, in particular an AAV or lentivirus vector.

33. The vector according to claim 31, which is an AAV vector comprising a capsid selected from the group consisting of: AAV1, AAV2, AAV5, AAV8, AAV2i8, AAV9, AAVrh10, AAVrh39, AAVrh43, AAVrh74, AAV-LK03, AAV2G9, AAV.PHP, AAV-Anc80, AAV3B capsids, and chimeric capsids thereof, in particular AAV8 capsid.

34. The vector according to claim 31, which is a particle or vesicle, in particular lipid-based micro- or nano-vesicle or particle comprising an RNA construct.

35. A cell comprising the vector according to claim 31.

36. The cell according to claim 35, wherein the cell is a muscle, liver or hematopoietic cell.

37. A pharmaceutical composition comprising at least an active agent selected from a nucleic acid construct according to claim 19, a vector comprising the nucleic acid construct, or a cell comprising the vector, and a pharmaceutically acceptable carrier.

38. A method of treatment of FGF-23 related hypophosphatemic diseases by gene therapy or cell therapy, comprising the administration of the pharmaceutical composition of claim 37 to a subject or cell of a subject in need thereof.

39. The method of treatment according to claim 38, wherein the FGF-23 related hypophosphatemic disease is a genetic disease selected from the group comprising: X-linked hypophosphatemia, Autosomal dominant hypophosphatemic rickets, Autosomal recessive hypophosphatemic rickets 1, Autosomal recessive hypophosphatemic rickets 2, Osteoglophonic dysplasia, Jansen type metaphyseal chondrodysplasia, Hypophosphatemia, dental anomalies and ectopic calcification, McCune-Albright syndrome/fibrous dysplasia, and Hypophosphatemia, skin and bone lesions, or an are acquired disease selected from the group comprising: Tumor-induced osteomalacia, Hypophosphatemic osteomalacia, complications from kidney transplantation or parenteral iron therapy, chronic kidney disease and its complications such as hyperparathyroidism, in particular X-linked hypophosphatemia.


SEQUENCE LISTING (58 sequences)

SEQ ID NO: 1 - protein, 251 aa, Homo sapiens
MLGARLRLWVCALCSVCSMSVLRAYPNASPLLGSSWGGLIHLYTATARNSYHLQIHKNGHVDGAPHQTIYSALMIRSEDAGFVVITGVMSRRYLCMDFRGNIFGSHYFDPENCRFQHQTLENGYDVYHSPQYHFLVSLGRAKRAFLPGMNPPPYSQFLSRRNEIPLIHFNTPIPRRHTRSAEDDSERDPLNVLKPRARMTPAPASCSQELPSAEDNSPMASDPLGVVRGGRVNTHAGGTGPEGCRPFAKFI

SEQ ID NO: 2 - protein, 77 aa, artificial sequence
  synthetic polypeptide (human FGF23 C-term peptide 175-251)
RRHTRSAEDDSERDPLNVLKPRARMTPAPASCSQELPSAEDNSPMASDPLGVVRGGRVNTHAGGTGPEGCRPFAKFI

SEQ ID NO: 3 - protein, 24 aa, artificial sequence
  synthetic peptide (human FGF23 signal peptide)
MLGARLRLWVCALCSVCSMSVLRA

SEQ ID NO: 4 - protein, 26 aa, artificial sequence
  synthetic peptide (alpha-1 anti-trypsin signal peptide)
MPSSVSWGILLLAGLCCLVPVSLAKL

SEQ ID NO: 5 - protein, 16 aa, artificial sequence
  synthetic peptide (synthetic mut1 signal peptide)
MLLLSALLLGLAFGYS

SEQ ID NO: 6 - protein, 16 aa, artificial sequence
  synthetic peptide (synthetic mut3 signal peptide)
MLLSFALLLGLALGYS

SEQ ID NO: 7 - protein, 18 aa, artificial sequence
  synthetic peptide (chymotrypsin B2 (CTRB2) signal peptide)
MAFLWLLSCWALLGTTFG

SEQ ID NO: 8 - protein, 22 aa, artificial sequence
  synthetic peptide (plasma protease C1 signal peptide)
MASRLTLLTLLLLLLAGDRASS

SEQ ID NO: 9 - protein, 585 aa, artificial sequence [sequence omitted]

SEQ ID NO: 10 - protein, 21 aa, artificial sequence
  synthetic peptide (FIX activation sequence (1))
SVSQTSKLTRAETVFPDVDGS

SEQ ID NO: 11 - protein, 18 aa, artificial sequence
  synthetic peptide (FIX activation sequence (2))
PVSQTSKLTRAETVFPDV

SEQ ID NO: 12 - protein, 701 aa, artificial sequence
  synthetic polypeptide
MAFLWLLSCWALLGTTFGRRHTRSAEDDSERDPLNVLKPRARMTPAPASCSQELPSAEDNSPMASDPLGVVRGGRVNTHAGGTGPEGCRPFAKFISVSQTSKLTRAETVFPDVDGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 13 - DNA, 2106 bp, artificial sequence
  synthetic polynucleotide codon optimized
atggctttcttgtggcttctcagttgttgggccttgttggggaccactttcggacgaagacacacacgctctgcagaggacgattctgagagggatcctttgaatgtccttaagccgagggcccgcatgacgcctgcaccagcatcctgcagtcaagagctcccttcagcagaagataatagtcctatggcgtccgaccccctgggggtagtacgcggggggcgagttaatacgcacgccgggggaactggtcctgaagggtgcaggccctttgccaagtttatttcagtgtcacaaacctctaaactgacacgcgccgagacggtctttccggatgttgatggatcagatgctcataaatcagaggtagctcaccgcttcaaagacctcggagaggagaactttaaggccctcgtcctcatcgcatttgcacagtatttgcaacaatgtccctttgaggatcatgtcaaattggtaaatgaagttactgagttcgcgaagacatgcgtggctgacgagtccgcagaaaactgcgacaagtcccttcatacactgtttggtgataaactgtgcactgtcgctactcttagagaaacatatggagagatggctgactgttgtgcgaagcaagagccagaacggaatgaatgtttccttcaacataaagatgataatccgaatttgccacgccttgtacgaccagaggtggatgttatgtgcaccgcgtttcatgacaatgaggaaacgttcctgaaaaaatatttgtatgagattgctcggagacatccttacttttacgcacccgagctcctgttcttcgctaagagatacaaggcagcctttacggaatgctgtcaggccgcggataaggccgcctgcttgctccccaagctggacgagctgcgagatgagggcaaggcgtcctctgctaagcaacgactcaaatgcgccagcttgcaaaagtttggcgaaagagcgtttaaggcctgggcagtggcgcgcctgagtcagcggttccctaaggcggagtttgccgaggtctcaaaactggtgacagacttgacgaaggtacataccgagtgctgccacggagatctcctggagtgcgcggatgatcgcgctgacctcgccaaatatatatgtgagaaccaggattccatttcctcaaaacttaaagagtgctgcgaaaaaccccttcttgagaagtcacactgcattgcagaggtcgaaaacgacgaaatgcctgccgatctgccttcccttgcggctgactttgtggagtctaaggacgtttgcaaaaactatgcagaggctaaagacgtattcctgggcatgtttctttatgaatacgcgcggcgacatcccgactattcagtcgtactcctgcttcgactggcgaaaacttacgaaactactctcgagaaatgctgtgctgccgccgaccctcatgagtgctatgctaaggtatttgatgaattcaaaccgttggttgaggaaccgcaaaacttgatcaagcagaactgtgaactttttgagcagctgggggaatataaattccagaatgcattgcttgtacgatacacaaagaaagttccacaggtctccaccccgacacttgtggaagttagcaggaacttgggaaaggtcggcagcaagtgttgtaagcaccctgaagctaagcgaatgccatgcgccgaagattacctttctgtggtgttgaaccaactgtgcgttcttcacgaaaagacgcccgtatctgatcgggtgacgaaatgctgcacagagtcacttgtgaacagacgcccctgctttagtgcgctcgaggtggacgaaacgtatgtcccgaaagagtttaacgcagagacattcacgtttcatgcggacatctgtaccctctctgaaaaggagagacagatcaaaaagcagacagcgctggttgagcttgttaagcataagccgaaggcgacaaaggagcaactgaaagcggtgatggatgacttcgccgcgttcgttgaaaaatgctgcaaagccgacgataaggaaacctgttttgctgaagaaggtaaaaaactcgttgctgcgagtcaggccgccctggggctgtga

SEQ ID NO: 14 - DNA, 397 bp, artificial sequence
  synthetic polynucleotide (hAAT promoter)
gatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaat

SEQ ID NO: 15 - DNA, 321 bp, artificial sequence
  synthetic polynucleotide (ApoE enhancer)
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggg

SEQ ID NO: 16 - DNA, 441 bp, artificial sequence
  synthetic polynucleotide (HBB2 intron)
gtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaag

SEQ ID NO: 17 - DNA, 441 bp, artificial sequence
  synthetic polynucleotide (modified HBB2 intron)
gtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttctggttgggataaggctggattattctgagtccaagctaggcccttttgctaatcttgttcatacctcttatcttcctcccacagctcctgggcaacctgctggtctctctgctggcccatcactttggcaaag

SEQ ID NO: 18 - DNA, 1438 bp, artificial sequence
  synthetic polynucleotide (FIX intron)
ggtttgtttccttttttaaaatacattgagtatgcttgccttttagatatagaaatatctgatgctgtcttcttcactaaattttgattacatgatttgacagcaatattgaagagtctaacagccagcacgcaggttggtaagtactggttctttgttagctaggttttcttcttcttcatttttaaaactaaatagatcgacaatgcttatgatgcatttatgtttaataaacactgttcagttcatgatttggtcatgtaattcctgttagaaaacattcatctccttggtttaaaaaaattaaaagtgggaaaacaaagaaatagcagaatatagtgaaaaaaaataaccacattatttttgtttggacttaccactttgaaatcaaaatgggaaacaaaagcacaaacaatggccttatttacacaaaaagtctgattttaagatatatgacatttcaaggtttcagaagtatgtaatgaggtgtgtctctaattttttaaattatatatcttcaatttaaagttttagttaaaacataaagattaacctttcattagcaagctgttagttatcaccaacgcttttcatggattaggaaaaaatcattttgtctctatgtcaaacatcttggagttgatatttggggaaacacaatactcagttgagttccctaggggagaaaagcacgcttaagaattgacataaagagtaggaagttagctaatgcaacatatatcactttgttttttcacaactacagtgactttatgtatttcccagaggaaggcatacagggaagaaattatcccatttggacaaacagcatgttctcacaggaagcatttatcacacttacttgtcaactttctagaatcaaatctagtagctgacagtaccaggatcaggggtgccaaccctaagcacccccagaaagctgactggccctgtggttcccactccagacatgatgtcagctgtgaaatcgacgtcgctggaccataattaggcttctgttcttcaggagacatttgttcaaagtcatttgggcaaccatattctgaaaacagcccagccagggtgatggatcactttgcaaagatcctcaatgagctattttcaagtgatgacaaagtgtgaagttaaccgctcatttgagaactttctttttcatccaaagtaaattcaaatatgattagaaatctgaccttttattactggaattctcttgactaaaagtaaaattgaattttaattcctaaatctccatgtgtatacagtactgtgggaacatcacagattttggctccatgccctaaagagaaattggctttcagattatttggattaaaaacaaagactttcttaagagatgtaaaattttcatgatgttttcttttttgctaaaactaaagaattattcttttacatttca

SEQ ID NO: 19 - DNA, 1438 bp, artificial sequence
  synthetic polynucleotide (modified FIX intron)
ggtttgtttccttttttaaaatacattgagtatgcttgccttttagatatagaaatatctgatgctgtcttcttcactaaattttgattacatgatttgacagcaatattgaagagtctaacagccagcacgcaggttggtaagtactggttctttgttagctaggttttcttcttcttcatttttaaaactaaatagatcgacattgcttttgttgcatttatgtttaataaacactgttcagttcatgatttggtcatgtaattcctgttagaaaacattcatctccttggtttaaaaaaattaaaagtgggaaaacaaagaaatagcagaatatagtgaaaaaaaataaccacattatttttgtttggacttaccactttgaaatcaaattgggaaacaaaagcacaaacaatggccttatttacacaaaaagtctgattttaagatatatgacatttcaaggtttcagaagtatgtaatgaggtgtgtctctaattttttaaattatatatcttcaatttaaagttttagttaaaacataaagattaacctttcattagcaagctgttagttatcaccaacgcttttcatggattaggaaaaaatcattttgtctctttgtcaaacatcttggagttgatatttggggaaacacaatactcagttgagttccctaggggagaaaagcacgcttaagaattgacataaagagtaggaagttagctattgcaacatatatcactttgttttttcacaactacagtgactttttgtatttcccagaggaaggcatacagggaagaaattatcccatttggacaaacagcttgttctcacaggaagcatttatcacacttacttgtcaactttctagaatcaaatctagtagctgacagtaccaggatcaggggtgccaaccctaagcacccccagaaagctgactggccctgtggttcccactccagacatgatgtcagctgtgaaatcgacgtcgctggaccataattaggcttctgttcttcaggagacatttgttcaaagtcatttgggcaaccatattctgaaaacagcccagccagggtgttggatcactttgcaaagatcctcattgagctattttcaagtgttgacaaagtgtgaagttaaccgctcatttgagaactttctttttcatccaaagtaaattcaaatatgattagaaatctgaccttttattactggaattctcttgactaaaagtaaaattgaattttaattcctaaatctccatgtgtatacagtactgtgggaacatcacagattttggctccatgccctaaagagaaattggctttcagattatttggattaaaaacaaagactttcttaagagatgtaaaattttcttgttgttttcttttttgctaaaactaaagaattattcttttacatttca

SEQ ID NO: 20 - DNA, 881 bp, artificial sequence
  synthetic polynucleotide (chicken beta globin intron)
gcgggagtcgctgcgttgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctc

SEQ ID NO: 21 - DNA, 881 bp, artificial sequence
  synthetic polynucleotide (Modified chicken beta-globin intron)
gcgggagtcgctgcgttgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttttggtaatcgtgcgagagggcgcagggacttcctttgtcccaaatctgtgcggagccgaaatctgggaggcgccgccgcaccccctctagcgggcgcggggcgaagcggtgcggcgccggcaggaaggaattgggcggggagggccttcgtgcgtcgccgcgccgccgtccccttctccctc

SEQ ID NO: 22 - DNA, 169 bp, artificial seqeunce
  synthetic polynucleotide (bGH polyA)
tgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtgggggctagctctaga

SEQ ID NO: 23 - DNA, 3598 bp, artificial sequence
  synthetic polynucleotide (expression cassette)
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatagatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttctggttgggataaggctggattattctgagtccaagctaggcccttttgctaatcttgttcatacctcttatcttcctcccacagctcctgggcaacctgctggtctctctgctggcccatcactttggcaaagcacgcgtgccaccatggctttcttgtggcttctcagttgttgggccttgttggggaccactttcggacgaagacacacacgctctgcagaggacgattctgagagggatcctttgaatgtccttaagccgagggcccgcatgacgcctgcaccagcatcctgcagtcaagagctcccttcagcagaagataatagtcctatggcgtccgaccccctgggggtagtacgcggggggcgagttaatacgcacgccgggggaactggtcctgaagggtgcaggccctttgccaagtttatttcagtgtcacaaacctctaaactgacacgcgccgagacggtctttccggatgttgatggatcagatgctcataaatcagaggtagctcaccgcttcaaagacctcggagaggagaactttaaggccctcgtcctcatcgcatttgcacagtatttgcaacaatgtccctttgaggatcatgtcaaattggtaaatgaagttactgagttcgcgaagacatgcgtggctgacgagtccgcagaaaactgcgacaagtcccttcatacactgtttggtgataaactgtgcactgtcgctactcttagagaaacatatggagagatggctgactgttgtgcgaagcaagagccagaacggaatgaatgtttccttcaacataaagatgataatccgaatttgccacgccttgtacgaccagaggtggatgttatgtgcaccgcgtttcatgacaatgaggaaacgttcctgaaaaaatatttgtatgagattgctcggagacatccttacttttacgcacccgagctcctgttcttcgctaagagatacaaggcagcctttacggaatgctgtcaggccgcggataaggccgcctgcttgctccccaagctggacgagctgcgagatgagggcaaggcgtcctctgctaagcaacgactcaaatgcgccagcttgcaaaagtttggcgaaagagcgtttaaggcctgggcagtggcgcgcctgagtcagcggttccctaaggcggagtttgccgaggtctcaaaactggtgacagacttgacgaaggtacataccgagtgctgccacggagatctcctggagtgcgcggatgatcgcgctgacctcgccaaatatatatgtgagaaccaggattccatttcctcaaaacttaaagagtgctgcgaaaaaccccttcttgagaagtcacactgcattgcagaggtcgaaaacgacgaaatgcctgccgatctgccttcccttgcggctgactttgtggagtctaaggacgtttgcaaaaactatgcagaggctaaagacgtattcctgggcatgtttctttatgaatacgcgcggcgacatcccgactattcagtcgtactcctgcttcgactggcgaaaacttacgaaactactctcgagaaatgctgtgctgccgccgaccctcatgagtgctatgctaaggtatttgatgaattcaaaccgttggttgaggaaccgcaaaacttgatcaagcagaactgtgaactttttgagcagctgggggaatataaattccagaatgcattgcttgtacgatacacaaagaaagttccacaggtctccaccccgacacttgtggaagttagcaggaacttgggaaaggtcggcagcaagtgttgtaagcaccctgaagctaagcgaatgccatgcgccgaagattacctttctgtggtgttgaaccaactgtgcgttcttcacgaaaagacgcccgtatctgatcgggtgacgaaatgctgcacagagtcacttgtgaacagacgcccctgctttagtgcgctcgaggtggacgaaacgtatgtcccgaaagagtttaacgcagagacattcacgtttcatgcggacatctgtaccctctctgaaaaggagagacagatcaaaaagcagacagcgctggttgagcttgttaagcataagccgaaggcgacaaaggagcaactgaaagcggtgatggatgacttcgccgcgttcgttgaaaaatgctgcaaagccgacgataaggaaacctgttttgctgaagaaggtaaaaaactcgttgctgcgagtcaggccgccctggggctgtgactcgagagatctaccggtgaattcaccgcgggtttaaactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtgggggctagctctaga

SEQ ID NO: 24 - DNA, 7559 bp, artificial sequence
  synthetic polynucleotide (AAV gene transfer vector pAAV hAAT sp7 FGF23-DNco)
cagcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagaggatcaaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatagatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttctggttgggataaggctggattattctgagtccaagctaggcccttttgctaatcttgttcatacctcttatcttcctcccacagctcctgggcaacctgctggtctctctgctggcccatcactttggcaaagcacgcgtgccaccatggctttcttgtggcttctcagttgttgggccttgttggggaccactttcggacgaagacacacacgctctgcagaggacgattctgagagggatcctttgaatgtccttaagccgagggcccgcatgacgcctgcaccagcatcctgcagtcaagagctcccttcagcagaagataatagtcctatggcgtccgaccccctgggggtagtacgcggggggcgagttaatacgcacgccgggggaactggtcctgaagggtgcaggccctttgccaagtttatttcagtgtcacaaacctctaaactgacacgcgccgagacggtctttccggatgttgatggatcagatgctcataaatcagaggtagctcaccgcttcaaagacctcggagaggagaactttaaggccctcgtcctcatcgcatttgcacagtatttgcaacaatgtccctttgaggatcatgtcaaattggtaaatgaagttactgagttcgcgaagacatgcgtggctgacgagtccgcagaaaactgcgacaagtcccttcatacactgtttggtgataaactgtgcactgtcgctactcttagagaaacatatggagagatggctgactgttgtgcgaagcaagagccagaacggaatgaatgtttccttcaacataaagatgataatccgaatttgccacgccttgtacgaccagaggtggatgttatgtgcaccgcgtttcatgacaatgaggaaacgttcctgaaaaaatatttgtatgagattgctcggagacatccttacttttacgcacccgagctcctgttcttcgctaagagatacaaggcagcctttacggaatgctgtcaggccgcggataaggccgcctgcttgctccccaagctggacgagctgcgagatgagggcaaggcgtcctctgctaagcaacgactcaaatgcgccagcttgcaaaagtttggcgaaagagcgtttaaggcctgggcagtggcgcgcctgagtcagcggttccctaaggcggagtttgccgaggtctcaaaactggtgacagacttgacgaaggtacataccgagtgctgccacggagatctcctggagtgcgcggatgatcgcgctgacctcgccaaatatatatgtgagaaccaggattccatttcctcaaaacttaaagagtgctgcgaaaaaccccttcttgagaagtcacactgcattgcagaggtcgaaaacgacgaaatgcctgccgatctgccttcccttgcggctgactttgtggagtctaaggacgtttgcaaaaactatgcagaggctaaagacgtattcctgggcatgtttctttatgaatacgcgcggcgacatcccgactattcagtcgtactcctgcttcgactggcgaaaacttacgaaactactctcgagaaatgctgtgctgccgccgaccctcatgagtgctatgctaaggtatttgatgaattcaaaccgttggttgaggaaccgcaaaacttgatcaagcagaactgtgaactttttgagcagctgggggaatataaattccagaatgcattgcttgtacgatacacaaagaaagttccacaggtctccaccccgacacttgtggaagttagcaggaacttgggaaaggtcggcagcaagtgttgtaagcaccctgaagctaagcgaatgccatgcgccgaagattacctttctgtggtgttgaaccaactgtgcgttcttcacgaaaagacgcccgtatctgatcgggtgacgaaatgctgcacagagtcacttgtgaacagacgcccctgctttagtgcgctcgaggtggacgaaacgtatgtcccgaaagagtttaacgcagagacattcacgtttcatgcggacatctgtaccctctctgaaaaggagagacagatcaaaaagcagacagcgctggttgagcttgttaagcataagccgaaggcgacaaaggagcaactgaaagcggtgatggatgacttcgccgcgttcgttgaaaaatgctgcaaagccgacgataaggaaacctgttttgctgaagaaggtaaaaaactcgttgctgcgagtcaggccgccctggggctgtgactcgagagatctaccggtgaattcaccgcgggtttaaactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtgggggctagctctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgtcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

SEQ ID NO: 25 - DNA, 768 bp, artificial sequence
  synthetic polynucleotide
  CDS (13)..(768)
acgcgtgccaccatgttgggggcccgcctcaggctctgggtctgtgccttgtgcagcgtctgcagcatgagcgtcctcagagcctatcccaatgcctccccactgctcggctccagctggggtggcctgatccacctgtacacagccacagccaggaacagctaccacctgcagatccacaagaatggccatgtggatggcgcaccccatcagaccatctacagtgccctgatgatcagatcagaggatgctggctttgtggtgattacaggtgtgatgagcagaagatacctctgcatggatttcagaggcaacatttttggatcacactatttcgacccggagaactgcaggttccaacaccagacgctggaaaacgggtacgacgtctaccactctcctcagtatcacttcctggtcagtctgggccgggcgaagagagccttcctgccaggcatgaacccacccccgtactcccagttcctgtcccggaggaacgagatccccctaattcacttcaacacccccataccacggcggcacacccggagcgccgaggacgactcggagcgggaccccctgaacgtgctgaagccccgggcccggatgaccccggccccggcctcctgttcacaggagctcccgagcgccgaggacaacagcccgatggccagtgacccattaggggtggtcaggggcggtcgagtgaacacgcacgctgggggaacgggcccggaaggctgccgccccttcgccaagttcatctag

SEQ ID NO: 26 - protein, 251 aa, artificial sequence; identical to SEQ ID NO: 1
  synthetic polynucleotide

SEQ ID NO: 27 - protein, 251 aa, artificial sequence; identical to SEQ ID NO: 1
  synthetic polypeptide

SEQ ID NO: 28 - DNA, 768 bp, artificial sequence
  synthetic polynucleotide
  CDS (13)..(768)
acgcgtgccaccatgttgggcgccaggcttcggctctgggtctgtgcactgtgcagtgtctgttccatgagtgtgttgcgggcttaccctaacgcatcaccgctgttgggctcttcctggggtggactcatacacctttatacggcgactgcgcgcaatagctaccatttgcaaatacacaagaatgggcatgttgatggtgctccccaccagactatctactctgctcttatgattaggtcagaagacgcggggttcgtagttattactggtgttatgtcccgcagatatctctgcatggacttccgcggtaacatctttgggtcacactatttcgacccagagaactgtcgcttccagcaccagacgttggagaatggttatgacgtgtatcactccccgcagtaccatttcttggtctctctgggccgggcgaaaagggcctttctgccaggtatgaatcctccgccttatagtcagttccttagcaggcggaacgaaattcctctgattcacttcaacacgccgatccctcgcaggcatacgcgatccgcagaagacgattctgagcgagatcctctcaatgttctgaaaccccgcgcccgaatgacgccagctccggccagttgtagtcaggagttgccgtctgccgaagataatagtcccatggcctccgatccacttggcgtggtaaggggaggtagagttaacacccacgccggtggaactggccctgaagggtgtcggccttttgccaaattcatatag

SEQ ID NO: 29 - protein, 251 aa, artificial sequence; identical to SEQ ID NO: 1
  Synthetic Construct

SEQ ID NO: 30 - DNA, 315 bp, artificial sequence
  synthetic polynucleotide
  CDS (13)..(315)
acgcgtgccaccatgttgggggcccgcctcaggctctgggtctgtgccttgtgcagcgtctgcagcatgagcgtcctcagagcccgaagacacacacgcgccgaggacgactcggagcgggaccccctgaacgtgctgaagccccgggcccggatgaccccggccccggcctcctgttcacaggagctcccgagcgccgaggacaacagcccgatggccagtgacccattaggggtggtcaggggcggtcgagtgaacacgcacgctgggggaacgggcccggaaggctgccgccccttcgccaagttcatctag

SEQ ID NO: 31 - protein, 100 aa, artificial sequence
  Synthetic Construct
MLGARLRLWVCALCSVCSMSVLRARRHTRAEDDSERDPLNVLKPRARMTPAPASCSQELPSAEDNSPMASDPLGVVRGGRVNTHAGGTGPEGCRPFAKFI

SEQ ID NO: 32 - protein, 100 aa, artificial sequence; identical to SEQ ID NO: 31
  synthetic polypeptide

SEQ ID NO: 33 - DNA, 300 bp, artificial sequence
  synthetic polynucleotide
  CDS (13)..(300)
acgcgtgccaccatgttgggggcccgcctcaggctctgggtctgtgccttgtgcagcgtctgcagcatgagcgtcctcagagccgccgaggacgactcggagcgggaccccctgaacgtgctgaagccccgggcccggatgaccccggccccggcctcctgttcacaggagctcccgagcgccgaggacaacagcccgatggccagtgacccattaggggtggtcaggggcggtcgagtgaacacgcacgctgggggaacgggcccggaaggctgccgccccttcgccaagttcatctag

SEQ ID NO: 34 - protein, 95 aa, artificial sequence
  Synthetic Construct
MLGARLRLWVCALCSVCSMSVLRAAEDDSERDPLNVLKPRARMTPAPASCSQELPSAEDNSPMASDPLGVVRGGRVNTHAGGTGPEGCRPFAKFI

SEQ ID NO: 35 - protein, 95 aa, artificial sequence; identical to SEQ ID NO: 34
  synthetic polypeptide

SEQ ID NO: 36 - DNA, 321 bp, artificial sequence
  synthetic polynucleotide
  CDS (13)..(321)
acgcgtgccaccatgttgggcgccaggcttcggctctgggtctgtgcactgtgcagtgtctgttccatgagtgtgttgcgggctcgaagacacacacgctctgcagaggacgattctgagagggatcctttgaatgtccttaagccgagggcccgcatgacgcctgcaccagcatcctgcagtcaagagctcccttcagcagaagataatagtcctatggcgtccgaccccctgggggtagtacgcggggggcgagttaatacgcacgccgggggaactggtcctgaagggtgcaggccctttgccaagtttattgtttaa

SEQ ID NO: 37 - protein, 102 aa, artificial sequence
  Synthetic Construct
MLGARLRLWVCALCSVCSMSVLRARRHTRSAEDDSERDPLNVLKPRARMTPAPASCSQELPSAEDNSPMASDPLGVVRGGRVNTHAGGTGPEGCRPFAKFIV

SEQ ID NO: 38 - protein, 102 aa, artificial sequence; identical to SEQ ID NO: 37
  synthetic polypeptide

SEQ ID NO: 39 - DNA, 306 bp, artificial sequence
  synthetic polynucleotide
  CDS (13)..(306)
acgcgtgccaccatgttgggcgccaggcttcggctctgggtctgtgcactgtgcagtgtctgttccatgagtgtgttgcgggcttctgcagaggacgattctgagagggatcctttgaatgtccttaagccgagggcccgcatgacgcctgcaccagcatcctgcagtcaagagctcccttcagcagaagataatagtcctatggcgtccgaccccctgggggtagtacgcggggggcgagttaatacgcacgccgggggaactggtcctgaagggtgcaggccctttgccaagtttattgtttaa

SEQ ID NO: 40 - protein, 97 aa, artificial sequence
  Synthetic Construct
MLGARLRLWVCALCSVCSMSVLRASAEDDSERDPLNVLKPRARMTPAPASCSQELPSAEDNSPMASDPLGVVRGGRVNTHAGGTGPEGCRPFAKFIV

SEQ ID NO: 41 - protein, 97 aa, artificial sequence; identical to SEQ ID NO: 40
  synthetic polypeptide

SEQ ID NO: 42 - DNA, 303 bp, artificial sequence
  synthetic polynucleotide
  CDS (13)..(303)
acgcgtgccaccatggctttcttgtggcttctcagttgttgggccttgttggggaccactttcggacgaagacacacacgctctgcagaggacgattctgagagggatcctttgaatgtccttaagccgagggcccgcatgacgcctgcaccagcatcctgcagtcaagagctcccttcagcagaagataatagtcctatggcgtccgaccccctgggggtagtacgcggggggcgagttaatacgcacgccgggggaactggtcctgaagggtgcaggccctttgccaagtttattgtttaa

SEQ ID NO: 43 - protein, 96 aa, artificial sequence
  Synthetic Construct
MAFLWLLSCWALLGTTFGRRHTRSAEDDSERDPLNVLKPRARMTPAPASCSQELPSAEDNSPMASDPLGVVRGGRVNTHAGGTGPEGCRPFAKFIV

SEQ ID NO: 44 - protein, 96 aa, artificial sequence; identical to SEQ ID NO: 43
  synthetic polypeptide

SEQ ID NO: 45 - DNA, 288 bp, artificial sequence
  synthetic polynucleotide
  CDS (13)..(288)
acgcgtgccaccatggctttcttgtggcttctcagttgttgggccttgttggggaccactttcggatctgcagaggacgattctgagagggatcctttgaatgtccttaagccgagggcccgcatgacgcctgcaccagcatcctgcagtcaagagctcccttcagcagaagataatagtcctatggcgtccgaccccctgggggtagtacgcggggggcgagttaatacgcacgccgggggaactggtcctgaagggtgcaggccctttgccaagtttattgtttaa

SEQ ID NO: 46 - protein, 91 aa, artificial sequence
  Synthetic Construct
MAFLWLLSCWALLGTTFGSAEDDSERDPLNVLKPRARMTPAPASCSQELPSAEDNSPMASDPLGVVRGGRVNTHAGGTGPEGCRPFAKFIV

SEQ ID NO: 47 - protein, 91 aa, artificial sequence; identical to SEQ ID NO: 46
  synthetic polypeptide

SEQ ID NO: 48 - DNA, 2055 bp, artificial sequence
  synthetic polynucleotide
  CDS (13)..(2055)
acgcgtgccaccatggctttcttgtggcttctcagttgttgggccttgttggggaccactttcggacgaagacacacacgctctgcagaggacgattctgagagggatcctttgaatgtccttaagccgagggcccgcatgacgcctgcaccagcatcctgcagtcaagagctcccttcagcagaagataatagtcctatggcgtccgaccccctgggggtagtacgcggggggcgagttaatacgcacgccgggggaactggtcctgaagggtgcaggccctttgccaagtttattgatgctcataaatcagaggtagctcaccgcttcaaagacctcggagaggagaactttaaggccctcgtcctcatcgcatttgcacagtatttgcaacaatgtccctttgaggatcatgtcaaattggtaaatgaagttactgagttcgcgaagacatgcgtggctgacgagtccgcagaaaactgcgacaagtcccttcatacactgtttggtgataaactgtgcactgtcgctactcttagagaaacatatggagagatggctgactgttgtgcgaagcaagagccagaacggaatgaatgtttccttcaacataaagatgataatccgaatttgccacgccttgtacgaccagaggtggatgttatgtgcaccgcgtttcatgacaatgaggaaacgttcctgaaaaaatatttgtatgagattgctcggagacatccttacttttacgcacccgagctcctgttcttcgctaagagatacaaggcagcctttacggaatgctgtcaggccgcggataaggccgcctgcttgctccccaagctggacgagctgcgagatgagggcaaggcgtcctctgctaagcaacgactcaaatgcgccagcttgcaaaagtttggcgaaagagcgtttaaggcctgggcagtggcgcgcctgagtcagcggttccctaaggcggagtttgccgaggtctcaaaactggtgacagacttgacgaaggtacataccgagtgctgccacggagatctcctggagtgcgcggatgatcgcgctgacctcgccaaatatatatgtgagaaccaggattccatttcctcaaaacttaaagagtgctgcgaaaaaccccttcttgagaagtcacactgcattgcagaggtcgaaaacgacgaaatgcctgccgatctgccttcccttgcggctgactttgtggagtctaaggacgtttgcaaaaactatgcagaggctaaagacgtattcctgggcatgtttctttatgaatacgcgcggcgacatcccgactattcagtcgtactcctgcttcgactggcgaaaacttacgaaactactctcgagaaatgctgtgctgccgccgaccctcatgagtgctatgctaaggtatttgatgaattcaaaccgttggttgaggaaccgcaaaacttgatcaagcagaactgtgaactttttgagcagctgggggaatataaattccagaatgcattgcttgtacgatacacaaagaaagttccacaggtctccaccccgacacttgtggaagttagcaggaacttgggaaaggtcggcagcaagtgttgtaagcaccctgaagctaagcgaatgccatgcgccgaagattacctttctgtggtgttgaaccaactgtgcgttcttcacgaaaagacgcccgtatctgatcgggtgacgaaatgctgcacagagtcacttgtgaacagacgcccctgctttagtgcgctcgaggtggacgaaacgtatgtcccgaaagagtttaacgcagagacattcacgtttcatgcggacatctgtaccctctctgaaaaggagagacagatcaaaaagcagacagcgctggttgagcttgttaagcataagccgaaggcgacaaaggagcaactgaaagcggtgatggatgacttcgccgcgttcgttgaaaaatgctgcaaagccgacgataaggaaacctgttttgctgaagaaggtaaaaaactcgttgctgcgagtcaggccgccctggggctgtga

SEQ ID NO: 49 - protein, 680 aa, artificial sequence
  Synthetic Construct
MAFLWLLSCWALLGTTFGRRHTRSAEDDSERDPLNVLKPRARMTPAPASCSQELPSAEDNSPMASDPLGVVRGGRVNTHAGGTGPEGCRPFAKFIDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 50 - protein, 680 aa, artificial sequence; identical to SEQ ID NO: 49
  synthetic polypeptide

SEQ ID NO: 51 - DNA, 2103 bp, artificial sequence
  synthetic polynucleotide
  CDS (13)..(2103)
acgcgtgccaccatggctttcttgtggcttctcagttgttgggccttgttggggaccactttcggatctgcagaggacgattctgagagggatcctttgaatgtccttaagccgagggcccgcatgacgcctgcaccagcatcctgcagtcaagagctcccttcagcagaagataatagtcctatggcgtccgaccccctgggggtagtacgcggggggcgagttaatacgcacgccgggggaactggtcctgaagggtgcaggccctttgccaagtttatttcagtgtcacaaacctctaaactgacacgcgccgagacggtctttccggatgttgatggatcagatgctcataaatcagaggtagctcaccgcttcaaagacctcggagaggagaactttaaggccctcgtcctcatcgcatttgcacagtatttgcaacaatgtccctttgaggatcatgtcaaattggtaaatgaagttactgagttcgcgaagacatgcgtggctgacgagtccgcagaaaactgcgacaagtcccttcatacactgtttggtgataaactgtgcactgtcgctactcttagagaaacatatggagagatggctgactgttgtgcgaagcaagagccagaacggaatgaatgtttccttcaacataaagatgataatccgaatttgccacgccttgtacgaccagaggtggatgttatgtgcaccgcgtttcatgacaatgaggaaacgttcctgaaaaaatatttgtatgagattgctcggagacatccttacttttacgcacccgagctcctgttcttcgctaagagatacaaggcagcctttacggaatgctgtcaggccgcggataaggccgcctgcttgctccccaagctggacgagctgcgagatgagggcaaggcgtcctctgctaagcaacgactcaaatgcgccagcttgcaaaagtttggcgaaagagcgtttaaggcctgggcagtggcgcgcctgagtcagcggttccctaaggcggagtttgccgaggtctcaaaactggtgacagacttgacgaaggtacataccgagtgctgccacggagatctcctggagtgcgcggatgatcgcgctgacctcgccaaatatatatgtgagaaccaggattccatttcctcaaaacttaaagagtgctgcgaaaaaccccttcttgagaagtcacactgcattgcagaggtcgaaaacgacgaaatgcctgccgatctgccttcccttgcggctgactttgtggagtctaaggacgtttgcaaaaactatgcagaggctaaagacgtattcctgggcatgtttctttatgaatacgcgcggcgacatcccgactattcagtcgtactcctgcttcgactggcgaaaacttacgaaactactctcgagaaatgctgtgctgccgccgaccctcatgagtgctatgctaaggtatttgatgaattcaaaccgttggttgaggaaccgcaaaacttgatcaagcagaactgtgaactttttgagcagctgggggaatataaattccagaatgcattgcttgtacgatacacaaagaaagttccacaggtctccaccccgacacttgtggaagttagcaggaacttgggaaaggtcggcagcaagtgttgtaagcaccctgaagctaagcgaatgccatgcgccgaagattacctttctgtggtgttgaaccaactgtgcgttcttcacgaaaagacgcccgtatctgatcgggtgacgaaatgctgcacagagtcacttgtgaacagacgcccctgctttagtgcgctcgaggtggacgaaacgtatgtcccgaaagagtttaacgcagagacattcacgtttcatgcggacatctgtaccctctctgaaaaggagagacagatcaaaaagcagacagcgctggttgagcttgttaagcataagccgaaggcgacaaaggagcaactgaaagcggtgatggatgacttcgccgcgttcgttgaaaaatgctgcaaagccgacgataaggaaacctgttttgctgaagaaggtaaaaaactcgttgctgcgagtcaggccgccctggggctgtga

SEQ ID NO: 52 - protein, 696 aa, artificial sequence
  Synthetic Construct
MAFLWLLSCWALLGTTFGSAEDDSERDPLNVLKPRARMTPAPASCSQELPSAEDNSPMASDPLGVVRGGRVNTHAGGTGPEGCRPFAKFISVSQTSKLTRAETVFPDVDGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 53 - protein, 696 aa, artificial sequence; identical to SEQ ID NO: 52
  synthetic polypeptide

SEQ ID NO: 54 - DNA, 2040 bp, artificial sequence
  synthetic polynucleotide
  CDS (13)..(2040)
acgcgtgccaccatggctttcttgtggcttctcagttgttgggccttgttggggaccactttcggatctgcagaggacgattctgagagggatcctttgaatgtccttaagccgagggcccgcatgacgcctgcaccagcatcctgcagtcaagagctcccttcagcagaagataatagtcctatggcgtccgaccccctgggggtagtacgcggggggcgagttaatacgcacgccgggggaactggtcctgaagggtgcaggccctttgccaagtttattgatgctcataaatcagaggtagctcaccgcttcaaagacctcggagaggagaactttaaggccctcgtcctcatcgcatttgcacagtatttgcaacaatgtccctttgaggatcatgtcaaattggtaaatgaagttactgagttcgcgaagacatgcgtggctgacgagtccgcagaaaactgcgacaagtcccttcatacactgtttggtgataaactgtgcactgtcgctactcttagagaaacatatggagagatggctgactgttgtgcgaagcaagagccagaacggaatgaatgtttccttcaacataaagatgataatccgaatttgccacgccttgtacgaccagaggtggatgttatgtgcaccgcgtttcatgacaatgaggaaacgttcctgaaaaaatatttgtatgagattgctcggagacatccttacttttacgcacccgagctcctgttcttcgctaagagatacaaggcagcctttacggaatgctgtcaggccgcggataaggccgcctgcttgctccccaagctggacgagctgcgagatgagggcaaggcgtcctctgctaagcaacgactcaaatgcgccagcttgcaaaagtttggcgaaagagcgtttaaggcctgggcagtggcgcgcctgagtcagcggttccctaaggcggagtttgccgaggtctcaaaactggtgacagacttgacgaaggtacataccgagtgctgccacggagatctcctggagtgcgcggatgatcgcgctgacctcgccaaatatatatgtgagaaccaggattccatttcctcaaaacttaaagagtgctgcgaaaaaccccttcttgagaagtcacactgcattgcagaggtcgaaaacgacgaaatgcctgccgatctgccttcccttgcggctgactttgtggagtctaaggacgtttgcaaaaactatgcagaggctaaagacgtattcctgggcatgtttctttatgaatacgcgcggcgacatcccgactattcagtcgtactcctgcttcgactggcgaaaacttacgaaactactctcgagaaatgctgtgctgccgccgaccctcatgagtgctatgctaaggtatttgatgaattcaaaccgttggttgaggaaccgcaaaacttgatcaagcagaactgtgaactttttgagcagctgggggaatataaattccagaatgcattgcttgtacgatacacaaagaaagttccacaggtctccaccccgacacttgtggaagttagcaggaacttgggaaaggtcggcagcaagtgttgtaagcaccctgaagctaagcgaatgccatgcgccgaagattacctttctgtggtgttgaaccaactgtgcgttcttcacgaaaagacgcccgtatctgatcgggtgacgaaatgctgcacagagtcacttgtgaacagacgcccctgctttagtgcgctcgaggtggacgaaacgtatgtcccgaaagagtttaacgcagagacattcacgtttcatgcggacatctgtaccctctctgaaaaggagagacagatcaaaaagcagacagcgctggttgagcttgttaagcataagccgaaggcgacaaaggagcaactgaaagcggtgatggatgacttcgccgcgttcgttgaaaaatgctgcaaagccgacgataaggaaacctgttttgctgaagaaggtaaaaaactcgttgctgcgagtcaggccgccctggggctgtga

SEQ ID NO: 55 - protein, 675 aa, artificial sequence
  Synthetic Construct
MAFLWLLSCWALLGTTFGSAEDDSERDPLNVLKPRARMTPAPASCSQELPSAEDNSPMASDPLGVVRGGRVNTHAGGTGPEGCRPFAKFIDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 56 - protein, 675 aa, artificial sequence; identical to SEQ ID NO: 55
  synthetic polypeptide

SEQ ID NO: 57 - DNA, 2118 bp, artificial sequence
  synthetic polynucleotide
  CDS (13)..(2118)
acgcgtgccaccatggctttcttgtggcttctcagttgttgggccttgttggggaccactttcggacgaagacacacacgctctgcagaggacgattctgagagggatcctttgaatgtccttaagccgagggcccgcatgacgcctgcaccagcatcctgcagtcaagagctcccttcagcagaagataatagtcctatggcgtccgaccccctgggggtagtacgcggggggcgagttaatacgcacgccgggggaactggtcctgaagggtgcaggccctttgccaagtttatttcagtgtcacaaacctctaaactgacacgcgccgagacggtctttccggatgttgatggatcagatgctcataaatcagaggtagctcaccgcttcaaagacctcggagaggagaactttaaggccctcgtcctcatcgcatttgcacagtatttgcaacaatgtccctttgaggatcatgtcaaattggtaaatgaagttactgagttcgcgaagacatgcgtggctgacgagtccgcagaaaactgcgacaagtcccttcatacactgtttggtgataaactgtgcactgtcgctactcttagagaaacatatggagagatggctgactgttgtgcgaagcaagagccagaacggaatgaatgtttccttcaacataaagatgataatccgaatttgccacgccttgtacgaccagaggtggatgttatgtgcaccgcgtttcatgacaatgaggaaacgttcctgaaaaaatatttgtatgagattgctcggagacatccttacttttacgcacccgagctcctgttcttcgctaagagatacaaggcagcctttacggaatgctgtcaggccgcggataaggccgcctgcttgctccccaagctggacgagctgcgagatgagggcaaggcgtcctctgctaagcaacgactcaaatgcgccagcttgcaaaagtttggcgaaagagcgtttaaggcctgggcagtggcgcgcctgagtcagcggttccctaaggcggagtttgccgaggtctcaaaactggtgacagacttgacgaaggtacataccgagtgctgccacggagatctcctggagtgcgcggatgatcgcgctgacctcgccaaatatatatgtgagaaccaggattccatttcctcaaaacttaaagagtgctgcgaaaaaccccttcttgagaagtcacactgcattgcagaggtcgaaaacgacgaaatgcctgccgatctgccttcccttgcggctgactttgtggagtctaaggacgtttgcaaaaactatgcagaggctaaagacgtattcctgggcatgtttctttatgaatacgcgcggcgacatcccgactattcagtcgtactcctgcttcgactggcgaaaacttacgaaactactctcgagaaatgctgtgctgccgccgaccctcatgagtgctatgctaaggtatttgatgaattcaaaccgttggttgaggaaccgcaaaacttgatcaagcagaactgtgaactttttgagcagctgggggaatataaattccagaatgcattgcttgtacgatacacaaagaaagttccacaggtctccaccccgacacttgtggaagttagcaggaacttgggaaaggtcggcagcaagtgttgtaagcaccctgaagctaagcgaatgccatgcgccgaagattacctttctgtggtgttgaaccaactgtgcgttcttcacgaaaagacgcccgtatctgatcgggtgacgaaatgctgcacagagtcacttgtgaacagacgcccctgctttagtgcgctcgaggtggacgaaacgtatgtcccgaaagagtttaacgcagagacattcacgtttcatgcggacatctgtaccctctctgaaaaggagagacagatcaaaaagcagacagcgctggttgagcttgttaagcataagccgaaggcgacaaaggagcaactgaaagcggtgatggatgacttcgccgcgttcgttgaaaaatgctgcaaagccgacgataaggaaacctgttttgctgaagaaggtaaaaaactcgttgctgcgagtcaggccgccctggggctgtga

SEQ ID NO: 58 - protein, 701 aa, artificial sequence; identical to SEQ ID NO: 12
  Synthetic Construct